(19) United States
(12) Patent Application Publication
(10) Pub. No.: US 2005/0238626 A1
(43) Pub. Date: Oct. 27, 2005

(54) Antigen specific T cell therapy

(76) Inventors: Lili Yang, Pasadena, CA (US); David Baltimore, Pasadena, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/071,785

(22) Filed: Mar. 2, 2005

Related U.S. Application Data
(60) Provisional application No. 60/558,663, filed on Apr. 1, 2004.
(60) Provisional application No. 60/571,811, filed on May 17, 2004.

(51) Int. Cl. A61K 48/00; C12N 5/08

(52) U.S. Cl. 424/93.21; 435/372

(57) ABSTRACT

Provided are methods for generating immune cells of the desired type and specificity in a host. The methods may be used to treat a disease or disorder, such as a tumor in a patient. Target cells, preferably hematopoietic stem cells such as primary bone marrow cells are transfected with a polynucleotide encoding a T cell receptor with the desired specificity. The transfected cells are then transferred to the host where they develop into mature, functional immune cells. The source of the T cell receptor can determine the stem cell's fate. Thus transfecting stem cells with TCRs from cytotoxic cells will lead to the generation of cytotoxic T cells in the host, while TCRs from helper cells will produce helper cells. Both arms of T cell immunity can be generated simultaneously in a host. Additionally, the immune response to the desired antigen can be further stimulated by immunizing the host with the antigen.

### REFERENCE TO RELATED APPLICATIONS

The present application claims priority to U.S. Provisional Application No. 60/558,663 filed Apr. 1, 2004 and U.S. Provisional Application No. 60/571,811, filed May 17, 2004. In addition, the present application is related to U.S. patent application Ser. Nos. 10/317,078, filed Dec. 10, 2002 and Ser. No. 10/789,938, filed Feb. 27, 2004.

### STATEMENT OF GOVERNMENT SUPPORT

This invention was made with government support under R01 GM39458 awarded by the National Institutes of Health. The government has certain rights in the invention.

### BACKGROUND OF THE INVENTION

1. Field of the Invention

The invention relates generally to the treatment of disease by the generation of antigen specific immune cells.

2. Description of the Related Art

The naturally occurring T cell repertoire in an individual is composed of up to 1×10¹² T cells expressing some 2.5×10⁷ T cell receptors (T cell receptors), with each T cell bearing T cell receptors of a single specificity. The enormous size of the T cell repertoire allows an animal to respond to a wide diversity of antigens. During an immune response, the antigen-specific T cell clones can rapidly expand. “Clonal expansion” is the hallmark of adaptive immunity, and provides an efficient way for the adaptive immune system to protect organisms against infectious diseases. While the immune system handles most pathogens well, it does a poor job of suppressing the growth of tumors. This phenomenon is not totally understood, but much evidence suggests that the limited number of T cells capable of responding to tumor cells, insufficient avidity of these T cells for tumor antigens, and tolerogenic attenuations by the tumor contribute to this immunological failure. Existing methods of cancer immunotherapy focus on reshaping the normal T cell repertoire, and fall into two categories: active expansion of the endogenous anti-tumor T cell clones by immunization, which involves activating the effectors in the host immune system to inhibit cancer cell growth and reject tumor (e.g. cancer vaccination) and passive immunotherapy, a term for directly providing the host with effectors to react against cancer (e.g., adoptive transfer of in vitro expanded or modified anti-tumor T cells).

A crucial step in the treatment of cancer with immunotherapy has been the identification of tumor antigens capable of stimulating T cell responses. Cytotoxic T cells (“CTLs,” “CD8 T cells”) have been shown to be the major effector cells that mediate tumor rejection. This has been supported by adoptive transfer studies in which CTL cell lines and CTL clones specific for tumor antigens, when activated in vitro, can mediate anti-tumor immunity. The role of CTLs in anti-tumor immunity is manifest by the fact that CTLs performed tumor killing upon direct recognition of tumor antigen peptides presented by the tumors MHC Class I molecules.

Recently, the other arm of T cell immunity, mediated by helper T cells (CD4 T cells), has attracted more and more attention. Accumulating evidence shows that CD4 T cells, which are known to play an essential role in organizing virtually all antigen-specific responses, also are critical in orchestrating multiple effector functions in anti-tumor immunity, including activation of CTLs, macrophages and eosinophils, and B cells. In addition, at least one cancer antigen has been shown to be recognized by CD8 and CD4 T cells, as well as antibodies, suggesting that CD4 T cells play a role in helping B cells to make anti-tumor antibodies. Further, the role of CD4 T cells and productive anti-tumor immunity has been demonstrated by the abrogation of anti-tumor immunity in CD4 knockout mice and mice depleted of CD4 T cells.

Currently, the only available method for the generation of an animal having a T cell with a defined antigen specificity is to introduce the gene encoding the desired T cell receptor into an embryo by pronuclear injection.

The introduction of a T cell receptor into peripheral blood cells has been reported recently (P. A. Moss (2001) Nature Immunology 2, 900-901; Kessels et al. (2001) Nature Immunology 2, 957-961 and Stanislawski et al. (2001) Nature Immunology 2, 962-970). In these studies, T cell receptor α and T cell receptor β genes were introduced and stably expressed in mature T cells that had been activated with a mitogen and then infected with a retroviral vector. Using this approach, T cells derived from non-specific, heterogeneous populations were converted into T cells capable of responding to protein antigens and tumor tissues. However, these methods do not produce lymphocytes having a specific antigen-specificity. Importantly, the T cells that are engineered to express the T cell receptors are activated mature cells that already express an endogenous T cell receptor of unknown specificity. Thus the introduction of transgenic T cell receptor α and β chains will lead to the heterologous combinations with the endogenous chains. These heterologous T cell receptors will have unpredictable specificity and may produce autoimmune damage. Furthermore, the effector function of the engineered cells is defined by the conditions under which these cells are activated in vitro, which will limit the type of immune responses they can induce.

### SUMMARY OF THE INVENTION

One aspect of the present invention relates to the generation of particular types of immune cells in a patient. In some embodiments, cytotoxic T cells are generated in the patient while in other embodiments helper T cells are generated in the patient. In still other embodiments, both types of T cells are generated. The T cells preferably express a preselected T cell receptor that is specific for an antigen of interest, typically an antigen associated with a disease or disorder from which the patient suffers.

In some embodiments, cytotoxic T cells are generated in a patient by transfecting target cells with a polynucleotide encoding a T cell receptor that is specific for a disease associated antigen and wherein the original source of the T cell receptor is a cytotoxic T cell. The target cells are transferred into the patient where they develop into cytotoxic T cells.

Similarly, helper T cells may be generated in a patient by transfecting target cells with a polynucleotide encoding a T cell receptor that is specific for a disease associated antigen, wherein the source of the T cell receptor is a helper T cell.

In one embodiment, populations of cytotoxic T cells and helper T cells that are responsive to a particular antigen are both generated in a mammal. A first population of hematopoietic stem cells is transfected with a first vector encoding the α and β chains of a first T cell receptor from a cytotoxic T cell. A second population of hematopoietic stem cells is transfected with a second vector encoding the α and β chains of a second T cell receptor from a helper T cell. The first and second populations of transfected cells are transferred to the mammal, producing populations of helper T cells and cytotoxic T cells that are responsive to the antigen of interest.

In another aspect, methods of treating a patient suffering from a disease or disorder, such as cancer or a viral infection, are provided. Target cells are provided that have been transfected with a vector encoding at least the α and β chains of a T cell receptor that is specific for an antigen associated with the disease to be treated. The target cells are preferably stem cells, more preferably hematopoietic stem cells such as primary bone marrow cells. The target cells are preferably obtained from the patient or an immunologically compatible donor.

The target cells are preferably transfected using a viral vector comprising the polynucleotide encoding the T cell receptor. The viral vector is preferably a retroviral vector, such as a lentiviral vector. The vector preferably comprises a first cDNA encoding the α chain of the T cell receptor and a second cDNA encoding the β chain of the T cell receptor. The first and second cDNAs are preferably separated by an IRES element.

The transfected cells are transferred into the patient where they develop into normal, functional T cells that are responsive to the antigen of interest.

In some embodiments the patient is immunized with the disease associated antigen. Immunizing may comprise injecting the patient with antigen presenting cells, such as dendritic cells, loaded with the tumor-associated antigen. Preferably the antigen presenting cells are obtained from the patient. In other embodiments, the patient is immunized by injection of the antigen. Immunization is preferably carried out at least one day following the transfer of the transfected cells into the patient, more preferably at least five days following transfer.

The T cell receptor may be from a cytotoxic T cell or a helper T cell, leading to the development of a population of cytotoxic T cells or helper T cells in the patient, respectively. In one embodiment, both arms of T cell immunity are generated. That is, both helper T cells and cytotoxic T cells capable of generating an immune response to the disease or disorder are generated in the patient.

In some embodiments, further T cell receptors that are specific for different disease associated antigens are identified and used to generate additional populations of T cells in the patient. In other embodiments, additional T cell receptors that are specific for different epitopes on the same disease associated antigen are identified and utilized to generate T cells in the patient.

In some embodiments, methods of treating a disease in a patient are provided. The disease may be, for example and without limitation, a cancer, such as a tumor, or a viral infection. A first population of target cells is provided that has been transfected with a polynucleotide encoding a first T cell receptor from a cytotoxic T cell. A second population of target cells is provided that has been transfected with a polynucleotide encoding a second T cell receptor from a helper T cell. The first and second T cell receptors are specific for an antigen associated with the disease.

In some embodiments a third population of target cells is provided that has been transfected with a polynucleotide encoding a third T cell receptor that is specific for a different antigen associated with the disease.

The target cells are preferably hematopoietic stem cells, more preferably primary bone marrow cells. In one embodiment they are obtained from the patient or an immunologically compatible donor.

The two or more populations of transfected target cells are transferred into the patient, such as by injection. In the patient the target cells give rise to at least two populations of T cells. In particular, as the first T cell receptor was originally identified from a cytotoxic T cell and the second T cell receptor was originally identified in a helper T cell, populations of cytotoxic T cells and helper T cells would be produced in the patient.

In some embodiments the patient is immunized with the disease associated antigen. In some embodiments the patient is injected with the antigen. Preferably, the antigen is loaded onto antigen presenting cells, such as dendritic cells, which are injected into the patient. The dendritic cells are preferably obtained from the patient. In one embodiment the antigen is obtained from the patient. In another embodiment the antigen is synthesized or purified from another source. The immunization is preferably carried out at least one day following transfer of the target cells into the patient, more preferably at least five days after transfer. The immunization may be repeated two or more times as desired.

In particular embodiments cytotoxic T cells and helper T cells that are responsive to an antigen of interest are generated in a mammal. A first population of hematopoietic stem cells, preferably primary bone marrow cells, are transfected with a construct comprising a polynucleotide encoding the α and β chains of a first T cell receptor from a cytotoxic T cell. A second population of stem cells is transfected with a second vector encoding the α and β chains of a second T cell receptor from a helper T cell. The first and second T cell receptors are preferably specific for the antigen of interest. Following transfection, the first and second populations of transfected T cells are preferably transferred to a mammal, where they are able to develop into populations of helper and cytotoxic T cells.

### BRIEF DESCRIPTION OF THE DRAWINGS

FIG. 1. Imparting the desired CD8 cytotoxic or CD4 helper T cell antigen specificity to the mouse T cell repertoire by retrovirus-mediated expression of T cell receptor (T cell receptor) cDNAs in hematopoietic stem cells (HSCs). HSCs from RAG1^{-/-} or B6 mice were infected with MOT1 or MOT2 retroviruses and transferred into either RAG1^{-/-} host mice (denoted as RAG1/MOT1 or RAG1/MOT2) or B6 host mice (denoted as B6/MOT1 or B6/MOT2), respectively. RAG1^{-/-} (denoted as RAG1) or B6 mice were included as controls. Seven weeks later, host mice were analyzed for OT1 and OT2 specific T cell development.

FIG. 1A is a schematic representation of the MOT1 and MOT2 retrovirus constructs (MSCV derived retrovirus expressing OT1 or OT2 T cell receptor cDNAs). MSCV: murine stem cell virus; LTR: long terminal repeat; IRES: internal ribosomal entry site; WRE: woodchuck responsive element.

FIG. 1B illustrates detection of HSCs expressing the OT1 T cell receptor transgenes in bone marrow (BM) of host mice by intracellular staining of T cell receptor Vα2 and Vβ5.

FIG. 1C illustrates thymic development of OT1 or OT2 T cells in host mice. Thymocytes expressing OT1 or OT2 T cell receptor transgenes were detected in host mice by intracellular T cell receptor Vα1 and Vβ5 staining (upper panel). The distribution of the developmental markers CD4 and CD8 on thymocytes is shown in the lower panel.

FIG. 1D illustrates detection of mature OT1 CD8 T cells or OT2 CD4 T cells in the spleen of host mice by FACS staining.

FIG. 2. Functional expression of T cell receptors (OT1 and OT2) in primary T cells to redirect their antigen specificity.

FIG. 2A. Surface expression of OT1 and OT2 T cell receptors in infected primary T cells. Spleen cells were infected with MOT1 and MOT2 and then stained with antibodies against T cell receptor Vα2 and Vβ5.1, 5.2. Cells infected with MIG retroviruses with no T cell receptor genes were used as a control. Primary CD4 T cells were harvested from B6 mice spleen and were stimulated in vitro with 0.5 μg/ml anti-CD3+0.5 μg/ml anti-CD28 for 3 days. On day 2, the cells were spin-infected with MOT1 or MOT2 retroviruses. On day 3, a fraction of cells was analyzed for surface expression of mouse T cell receptor Vα2 and Vβ5.1, 5.2 by FACS.

FIG. 2B. Functional expression of OT1 and OT2 T cell receptors in primary T cells. The MOT1- and MOT2-infected cells were stimulated by their cognate antigens OVAp1 and OVAp2 in the presence of B6 spleen cells as antigen presenting cells (APCs). The infected cells responded to stimulation as measured by IFN-γ production using ELISA.

FIG. 3. Comparison of the OT2 T cell receptor expression and T cell development in mice receiving retrovirus-transduced RAG1 deficient HSCs with those in the conventional T cell receptor transgenic mice. RAG1 mice were included as a control.

FIG. 3A. OT2 T cell receptor expression in BM derived from OT2RAG1^{-/-} transgenic mice (denoted as OT2/RAG1 Tg) and RAG1/MOT2 mice. Intracellular staining was used to evaluate the expression level.

FIG. 3B. OT2 T cell expression and T cell development in the thymus. Thymocytes were harvested from OT2/RAG1 Tg and RAG1/MOT2 mice. T cell development was assessed by co-staining CD4 and CD8 surface markers. OT2 T cell receptor α and β chain expression was measured by intracellular staining. Expression at each different developmental stage (DN, DP and CD4 SP) is shown.

FIG. 3C. OT2 T cell receptor expression in spleen OT2 CD4 T cells derived from unchallenged and immunized OT2/RAG1 Tg and RAG1/MOT2 mice. Mice were immunized with OVAp2 antigen and CFA for 6 days. Both intracellular and surface expression of OT2 T cell receptor were measured.

FIG. 3D. OT2 T cell numbers in unchallenged (naïve) and immunized mice. OT2 T cells were identified by Vα2 and Vβ5.1, 5.2 surface staining.

FIG. 4 Characterization of the OT1 CD8 or OT2 CD4 T cells generated by retroviral transduction of wild-type B6 HSCs. OT1 or OT2 T cells harvested from B6/MOT1 or B6/MOT2 host mice 8 weeks after BM transfer were considered to be naïve. They were stimulated with OVAp₂₅₇₋₂₆₉ (denoted as OVAp1) or OVAp₃₂₉₋₃₃₇ (denoted as OVAp2) in vitro for 3 days to generate effector OT1 or OT2 T cells, which were then transferred into RAG1^{-/-} recipient mice. Fourteen or sixteen weeks later, the recipient mice were analyzed for the presence of memory OT1 or OT2 T cells.

FIG. 4A shows patterns of surface activation markers on OT1 T cells at the naïve, effector or memory stages measured by FACS staining. Surface markers studied are indicated below each column of results.

FIG. 4B presents a functional analysis of the naïve OT1 T cells (denoted as OT1(BMT). Proliferation (left) and IFN-γ production (right) in response to OVAp1 stimulation are shown. The responses were compared with those of conventional transgenic OT1 T cells (denoted as OT1(Tg)). B6 spleen cells were included as a negative control (B6 Ctrl).

FIG. 4C presents a functional analysis of memory OT1 T cells. Dosage response (left) and time-course response (middle) of OT1 memory T cells to OVAp1 stimulation measured by IFN-γ production, and proliferation response to cytokine stimulation (right) are shown. The responses were compared with those of the naïve OT1 T cells. B6 spleen cells were included as a negative control.

FIG. 5A shows patterns of surface activation markers on OT2 T cells at the naïve, effector or memory stages measured by FACS staining. Surface markers studied are indicated below each column of results.

FIG. 5B presents a functional analysis of the naïve OT2 T cells (denoted as OT2(BMT). Proliferation (left) and IL-2 production (right) in response to OVAp2 stimulation, and proliferation response to cytokine stimulation (bottom) are shown. The responses were compared with those of conventional transgenic OT2 T cells (denoted as OT2(Tg)). B6 spleen cells were included as a negative control (B6 Ctrl).

FIG. 5C illustrates the dosage response (upper panels) and time-course response (lower panels) of OT2 memory T cells to OVAp2 stimulation measured by IL-2, IL-4 and IFN-γ production. The responses were compared to those of naïve OT1 or OT2 T cells and B6 spleen cells were included as a negative control.

FIG. 6. Eradication of long-established large solid tumors by construction of both arms of the anti-tumor T cell immunity. E.G7 tumor cells were used.

FIG. 6A Detection of mature OT1 and OT2 T cells in the periphery of B6 mice receiving both B6 HSCs transduced with MOT1 retroviruses and B6 HSCs transduced with MOT2 retroviruses (denoted as B6/MOT1+MOT2). FACS analysis of the spleen cells is shown.

FIG. 6B illustrates the effect of different treatments on solid tumor growth in mice. Tumor size is shown as the product of the two largest perpendicular diameters a×b (mm²). Mice were euthanized when the tumors reached 400 mm².

FIG. 6C. Solid tumor growth in mice receiving different treatments is shown. Solid tumor size is given as the product of the two largest perpendicular diameters a×b (mm²). Mice were euthanized when the tumors reached 400 mm².

### DETAILED DESCRIPTION OF THE PREFERRED EMBODIMENT

Methods are provided for generating immune cells with a desired antigen specificity. These methods can be used to treat a wide variety of diseases and disorders. Although primarily described in terms of cancer therapy, one of skill in the art will recognize that the methods can be applied to the treatment of other diseases and disorders for which associated antigens can be identified, such as viral infections.

In some embodiments of these methods, cDNAs encoding a T cell receptor (TCR) with the desired specificity are introduced into hematopoietic stem cells (HSCs). Preferably a viral delivery system is used to transfect the cells, such as a retroviral vector system. Typically, the encoded T cell receptor is specific for a particular disease-associated antigen. The virus-transduced HSCs are then transferred into the host where they efficiently give rise to T cells with the desired specificity in vivo. By taking advantage of the HSC characteristics of longevity and self-renewal, this method provides the host with a lifelong supply of highly disease specific T cells in large quantity.

These methods not only allow for the generation of cells producing particular TCRs with specificity for particular antigens, but also allow for the selection of the type of T cell (e.g., cytotoxic T cell or helper T cell) which will develop in the host. Generally, selection of the type of T cell can be achieved by selecting a TCR that is from the cell type to be generated. For example, use of a TCR that is obtained from a cytotoxic T cell will result in the production of cytotoxic T cells in the host. Similarly, using a TCR whose origin is helper T cells will result in the production of CD4 helper cells in the host. Thus, the source of the TCR to be used for cell transfection can determine not only what antigen the resulting immune cells can recognize through their TCRs, but will also determine the fate of the cells.

In some embodiments, even though the cell fate is determined, a population of cells is maintained in the host that possesses characteristics of a stem cell, such as self-renewal and longevity. That is, although transfected stem cells will produce immune cells with the desired specificity, a population of precursor cells will be maintained that will provide the host with a lifetime supply of the desired type of immune cells.

The methods may be used therapeutically to generate a desired immune response in a patient in need of treatment. The treatment can be combined with other therapeutic methods, such as vaccination. Thus, in some embodiments immune cells are generated through the disclosed TCR dependent processes and are effective in treating a patient suffering from a disease or disorder. For example, a T cell receptor that binds to a particular antigen associated with a patient's tumor can be used to generate immune cells in the patient that express only the precise T cell receptor needed to generate an effective immune response against the tumor. Additionally, as the nature or source of the T cell receptor can determine the type of immune cells that develop in the host, the desired cell type can also be produced in the patient. For example, helper T cells, cytotoxic T cells or both can be generated in the patient. Thus, an immune response can be generated to particular targets in a patient, utilizing a particular arm of the immune system.

In some embodiments, both CD8 cytotoxic T cells (CTLs) and CD4 helper T cells dare generated in one host, providing synergistic benefits through the collaboration of both arms of the T cell immunity.

In some embodiments, the host may further be immunized with the antigen recognized by the selected TCR. The immunization stimulates the immune response to the target antigen and leads to an even greater degree of efficacy in treating the disease or disorder. The immunization may be repeated multiple times to obtain maximal results.

The methods disclosed herein can be used to prevent, treat or slow the progression of a disease or disorder. For example, the methods may be used to prevent the formation of a tumor, or reduce or eliminate a tumor that is already present in a patient.

In other embodiments, a variety of T cells with specificity to a number of different disease antigens or different epitopes on a single antigen are generated in the host. However, each T cell that is generated remains specific for a single antigen and expresses a single type of TCR. The targeting of multiple antigens and/or epitopes can reduce the likelihood that epitope escape will occur and that a disease will progress in spite of the treatment.

A. Definitions

Unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). Any methods, devices and materials similar or equivalent to those described herein can be used in the practice of this invention.

As used herein, the terms nucleic acid, polynucleotide and nucleotide are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages.

The terms nucleic acid, polynucleotide and nucleotide also specifically include nucleic acids composed of bases other than the five biologically occurring bases (adenine, guanine, thymine, cytosine and uracil).

As used herein, a nucleic acid molecule is said to be “isolated” when the nucleic acid molecule is substantially separated from contaminant nucleic acid molecules encoding other polypeptides.

“Immunization” refers to the provision of antigen to a host. The antigen is preferably an antigen that is recognized by T cells that have been generated in the host as disclosed herein. In the preferred embodiments, antigen is loaded onto antigen-presenting cells, such as dendritic cells, which are subsequently administered to the recipient, as described in more detail below. Other methods of immunization are well known in the art and may be used.

An “antigen” is any molecule that is capable of binding to a T cell receptor. Preferred antigens those that are capable of initiating an immune response upon binding to a T cell receptor that is expressed in an immune cell. An “immune response” is any biological activity that is attributable to the binding of an antigen to a T cell receptor.

The term “epitope” is used to refer to a site on an antigen that is recognized by a T cell receptor.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas.

“Native antibodies” and “native immunoglobulins” are usually heterotetrameric glycoproteins, composed of two identical light (L) chains and two identical heavy (H) chains. Each light chain is linked to a heavy chain by a disulfide bond. The number of disulfide linkages varies among the heavy chains of different immunoglobulin isotypes. Each heavy chain comprises a variable domain (V_{H}) followed by a number of constant domains. Each light chain comprises a variable domain at one end (V_{L}) and a constant domain at its other end. The constant domain of the light chain is aligned with the first constant domain of the heavy chain, and the light-chain variable domain is aligned with the variable domain of the heavy chain.

The term “antibody” is used in the broadest sense and specifically covers human, non-human (e.g. murine) and humanized monoclonal antibodies (including full length monoclonal antibodies), polyclonal antibodies, multi-specific antibodies (e.g., bispecific antibodies), and antibody fragments so long as they exhibit the desired biological activity.

As used herein, the term “T cell receptor” includes a complex of polypeptides comprising at least a T cell receptor α subunit and a T cell receptor β subunit. T cell receptors (“TCRs”) are able to bind antigen when expressed on the surface of a cell, such as a T lymphocyte. The α and β chains, or subunits, form a dimer that is independently capable of antigen binding. The α and β subunits typically comprise a constant domain and a variable domain and may be native, full-length polypeptides, or may be modified in some way, provided that the T cell receptor retains the ability to bind antigen. For example, the α and β subunits may be amino acid sequence variants, including substitution, addition and deletion mutants. They may also be chimeric subunits that comprise, for example, the variable regions from one organism and the constant regions from a different organism.

“Target cells” are any cells that are capable of expressing an antigen-specific polypeptide on their surface or that can mature into cells that express an antigen specific polypeptide, such as a T cell receptor, on their surface. Preferably, target cells are capable of maturing into immune cells, such as lymphocytes. Target cells include, without limitation, stem cells, particularly hematopoietic stem cells.

As used herein, a cell exhibits a “unique antigen specificity” if it is primarily responsive to a single type of antigen.

The term “mammal” is defined as an individual belonging to the class Mammalia and includes, without limitation, humans, domestic and farm animals, and zoo, sports, and pet animals, such as sheep, dogs, horses, cats and cows.

A “subject” or “patient” is any animal, preferably a mammal, that is in need of treatment.

As used herein, “treatment” is a clinical intervention made in response to a disease, disorder or physiological condition manifested by a patient or to be prevented in a patient. The aim of treatment includes the alleviation and/or prevention of symptoms, as well as slowing, stopping or reversing the progression of a disease, disorder, or condition. “Treatment” refers to both therapeutic treatment and prophylactic or preventative measures. Those in need of treatment include those already affected by a disease or disorder or undesired physiological condition as well as those in which the disease or disorder or undesired physiological condition is to be prevented. “Treatment” need not completely eliminate a disease, nor need it completely prevent a subject from catching the disease or disorder.

“Tumor,” as used herein, refers to all neoplastic cell growth and proliferation, whether malignant or benign, and all pre-cancerous and cancerous cells and tissues.

The term “cancer” refers to a disease or disorder that is characterized by unregulated cell growth. Examples of cancer include, but are not limited to, carcinoma, lymphoma, blastoma and sarcoma. Examples of specific cancers include, but are not limited to, lung cancer, colon cancer, breast cancer, testicular cancer, stomach cancer, pancreatic cancer, ovarian cancer, liver cancer, bladder cancer, colorectal cancer, and prostate cancer. Additional cancers are well known to those of skill in the art.

A “vector” is a nucleic acid molecule that is capable of transporting another nucleic acid. Vectors may be, for example, plasmids, cosmids or phage. An “expression vector” is a vector that is capable of directing the expression of a protein encoded by one or more genes carried by the vector when it is present in the appropriate environment.

The term “regulatory element” and “expression control element” are used interchangeably and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells may include, without limitation, promoters, enhancers, splicing signals and polyadenylation signals.

The term “transfection” refers to the introduction of a nucleic acid into a host cell.

By “transgene” is meant any nucleotide or DNA sequence that is integrated into one or more chromosomes of a target cell by human intervention. In one embodiment the transgene comprises a polynucleotide that encodes a T cell receptor whose expression in an immune cell is desired. The polynucleotide is generally operatively linked to other sequences that are useful for obtaining the desired expression of the gene of interest, such as transcriptional regulatory sequences. In other embodiments the transgene can comprise additional polynucleotide sequences, such as DNA that is used to mark the chromosome where the transgene has integrated.

The term “transgenic” is used herein to describe the property of harboring a transgene. For instance, a “transgenic organism” is any animal, including mammals, fish, birds and amphibians, in which one or more of the cells of the animal contain nucleic acid introduced by way of human intervention. In the typical transgenic animal, the transgene causes expression of a recombinant protein.

“Retroviruses” are enveloped RNA viruses that are capable of infecting animal cells. “Lentivirus” refers to a genus of retroviruses that are capable of infecting both dividing and non-dividing cells. Several examples of lentiviruses include HIV (human immunodeficiency virus; including HIV type 1, and HIV type 2), visna-maedi, the caprine arthritis-encephalitis virus, equine infectious anemia virus, feline immunodeficiency virus (FIV), bovine immune deficiency virus (BIV), and simian immunodeficiency virus (SIV).

“Transformation,” as defined herein, describes a process by which exogenous DNA enters a target cell. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and particle bombardment. “Transformed” cells include stably transformed cells in which the inserted DNA is capable of replication either as an autonomously replicating plasmid or as part of the host chromosome. Also included are cells that transiently express the antigen specific polypeptide.

Therapy

The methods of the present invention can be used to prevent or treat a disease or disorder for which an associated antigen can be identified. Diseases or disorders that are amenable to treatment or prevention by the methods of the present invention include, without limitation, cancers, autoimmune diseases, and infections, including viral, bacterial, fungal and parasitic infections.

In one embodiment a patient is suffering from a disease or disorder that is to be treated. An antigen that is associated with the disease or disorder is identified. Antigens associated with many diseases and disorders are well known in the art. Thus, the antigen may be previously known to be associated with the disease or disorder, or may be identified by any method known in the art. For example, an antigen to a type of cancer from which a patient is suffering may be known, such as a tumor associated antigen. Tumor associated antigens are not limited in any way and include, for example, antigens that are identified on cancerous cells from the patient to be treated.

Tumor associated antigens are known for a variety of cancers including, for example, prostate cancer and breast cancer. In some breast cancers, for example, the Her-2 receptor is overexpressed on the surface of cancerous cells. A number of tumor associated antigens have been reviewed (see, for example, “Tumor-Antigens Recognized By T-Lymphocytes,” Boon T, Cerottini J C, Vandeneynde B, Vanderbruggen P, Vanpel A, Annual Review Of Immunology 12: 337-365, 1994; “A listing of human tumor antigens recognized by T cells,” Renkvist N, Castelli C, Robbins P F, Parmiani G. Cancer Immunology Immunotherapy 50: (1) 3-15 MAR 2001).

In other embodiments, an antigen related to the disease or disorder is identified from the patient to be treated. For example, an antigen associated with a tumor may be identified from the tumor itself by any method known in the art.

Once an antigen has been identified and/or selected, one or more T cell receptors that are specific for the antigen are then identified. If a T cell receptor specific for the identified disease-associated antigen is not already known, it may be identified by any method known in the art. Identification of T cell receptors is discussed in detail below. T cell receptors may be identified from cytotoxic T cells, from helper T cells, or both, depending on the type of immune cell that is to be generated in the patient. For example, if cytotoxic T cells are to be generated in the patient, the T cell receptor is identified from a CTL. On the other hand, if helper T cells are to be generated, the T cell receptor is identified from a helper T cell. As discussed below, in some embodiments a T cell receptor from a CTL and a T cell receptor from a helper T cell are both utilized.

A polynucleotide that encodes the desired T cell receptor is identified and inserted in target cells. Preferably the polynucleotide comprises a cDNA that encodes the T cell receptor α subunit and a cDNA that encodes the T cell receptor β subunit. The T cell receptor polynucleotide is preferably inserted into a vector that is used to transfect target cells. In the preferred embodiments the vector is a retroviral vector. In some embodiments, the retroviral vector preferably comprises a modified lentivirus that is able to infect non-dividing cells, thus avoiding the need for in vitro propagation of the target cells. Virus is produced from the vector and used to infect target cells.

The target cells preferably comprise hematopoietic stem cells, more preferably bone marrow stem cells. In the preferred embodiment, the target cells are obtained from the mammal to be treated. Methods for obtaining bone marrow stem cells are well known in the art. In other embodiments, target cells are obtained from a donor, preferably an immunologically compatible donor.

In one particular embodiment, target cells are hematopoietic stem cells removed from a cancer patient prior to chemotherapy.

Following transfection of the target cells with the T cell receptor polynucleotide, the target cells are reconstituted in the mammal according to any method known in the art, such as by injection, where they mature into functional immune cells.

In a preferred embodiment the methods of the present invention are used to treat a patient suffering from cancer, such as a tumor. An antigen associated with the cancer is identified and one or more T cell receptors that recognize the antigen are obtained. A polynucleotide that encodes the T cell receptor is cloned.

Target cells, preferably hematopoietic stem cells, more preferably primary bone marrow cells, are obtained and transfected with the T cell receptor. The target cells are preferably obtained from the patient, but may be obtained from another source, such as an immunologically compatible donor.

The polynucleotide encoding the T cell receptor is preferably introduced into the target cells using a modified retrovirus, more preferably a modified lentivirus. The target cells are then transferred back to the patient, for example by injection, where they develop into immune cells that are capable of generating an immune response when contacted with the identified antigen. As demonstrated in the Examples below, the resulting immune cells generated in the patient express the particular TCR and the patient is able to mount an effective immune response against the disease or disorder.

In some embodiments the T cell receptor is cloned from cytotoxic T cells. This results in the generation of cytotoxic T cells in the patient, as discussed in more detail below. In other embodiments the T cell receptor is cloned from a helper T cell, resulting in the generation of helper T cells in the patient.

In still other embodiments both types of T cells are generated in the patient. The population of target cells is divided and some stem cells are transfected with a vector encoding a T cell receptor obtained from a cytotoxic T cell and some stem cells are transfected with a vector encoding a T cell receptor obtained from a helper T cell. The target stem cells are transferred into the patient, resulting in the simultaneous generation of a population of helper T cells specific for the disease or disorder and a population of cytotoxic T cells specific for the disease or disorder in the patient.

Additionally, transfecting different target cells with TCRs to different disease associated antigens or different epitopes of the same antigen can reduce the risk of epitope escape. Thus in some embodiments one population of target cells is transfected with a TCR specific to one antigen or epitope on an antigen and a second population of target cells is transfected with a TCR specific to a second antigen or a second epitope on the same antigen. Additional populations may be transfected with additional TCRs as desired. Upon transfer into the patient, each population of target cells gives rise to a population of immune cells with the desired specificity. In this way, a multi-pronged immune response to multiple disease-associated antigens can be generated in the patient.

In other embodiments, a disease or disorder is prevented from developing in a mammal. An antigen is identified that is associated with the disease or disorder that is expected to develop or to which the mammal is likely to be exposed. For example, if the disease or disorder is an infection, an antigen is identified that is associated with the infectious agent. Antigens for infectious agents, such as viruses and bacteria are known in the art or can be identified using known methods.

In one embodiment, a mammal has been or is expected to be exposed to an infectious agent, such as an infectious bacteria or virus, for example HIV. An antigen present on the infectious agent is identified. A polynucleotide that encodes an antigen-specific polypeptide, preferably a T cell receptor that is specific for that antigen, is cloned. Hematopoietic stem cells, preferably bone marrow stem cells, are contacted with a modified retrovirus that comprises the antigen-specific polynucleotide. Preferably the stem cells are obtained from the individual that is expected to be exposed to the infectious agent. Alternatively, they are obtained from another mammal, preferably an immunologically compatible donor. The transfected cells are then transferred into the individual where they develop into mature T cells that are capable of generating an immune response when presented with the antigen from the infectious agent. As discussed above, the T cell receptor may be obtained from cytotoxic T cells, helper T cells, or both, resulting in the generation of the respective types of T cells in the patient.

Identification of T Cell Receptors

Once an antigen associated with the disease to be treated has been identified and/or selected, a T cell receptor that is capable of interacting with the antigen is identified, along with a polynucleotide that encodes it. The T cell receptor may be identified from a cytotoxic T cell (CTL) or from a helper T cell. In some embodiments, T cell receptors are identified from both CTLs and helper T cells.

CTLs and helper T cells may be identified based on their expression of well known markers. In particular, CTLs may be identified based on expression of CD8, while helper T cells may be identified by expression of CD4. As discussed below, use of a T cell receptor identified from a CTL in the methods disclosed herein will lead to the production of CTLs in the host, while the use of a T cell receptor from a helper T cell will result in the production of helper T cells in the hose.

As used herein, the term “polynucleotide” may include more than one molecule. Thus, the polynucleotide encoding the T cell receptor may comprise two or more independent polynucleotide molecules, each encoding a distinct subunit. In other embodiments, all of the subunits may be encoded by a single polynucleotide. For example, the polynucleotide may comprise a first polynucleotide encoding the α subunit and a second polynucleotide encoding the β subunit of a T cell receptor.

The polynucleotide encoding the T cell receptor can be derived from any source, but is preferably derived from genomic DNA or from cDNA. In addition, the polynucleotide encoding the T cell receptor can be produced synthetically or isolated from a natural source. The polynucleotide may comprise, without limitation, DNA, cDNA and/or RNA sequences. Preferably, the polynucleotide comprises a cDNA encoding the α subunit of a T cell receptor and a cDNA encoding the β subunit of a T cell receptor.

It is understood that all polynucleotides encoding a desired T cell receptor are included herein. Such polynucleotides include, without limitation, naturally occurring, synthetic, and intentionally manipulated polynucleotides. For example, the polynucleotide may be a naturally occurring polynucleotide that has been subjected to site-directed mutagenesis. Also included are naturally occurring polynucleotides that comprise deletions insertions or substitutions, so long as they encode T cell receptors that retain the ability to interact with the desired antigen.

The polynucleotides may also be sequences that are degenerate as a result of the genetic code. There are 20 natural amino acids, most of which are specified by more than one codon. Therefore, all degenerate nucleotide sequences are included in the invention as long as the encoded polypeptide has the desired specificity.

In one embodiment, the polynucleotide sequence is a cDNA sequence. In another embodiment, the polynucleotide sequence is a cDNA sequence that has been intentionally manipulated, such as a cDNA that has been mutated to remove potential splice sites or to match codon usage to a particular host organism. Such manipulations are within the ordinary skill in the art.

The T cell receptor that is to be expressed in the immune cells produced in the host is preferably obtained from the same species as the host in which an immune response is to be generated. For example, when an immune response is to be generated in a human patient suffering from cancer, one or more human T cell receptors specific for an appropriate cancer antigen are identified.

When a T cell receptor sequence is determined in an organism other than that in which the immune response is to be generated, a chimeric T cell receptor is preferably used comprising the variable regions of the T cell receptor from the donor organism and the constant regions from T cell receptors from the organism in which the immune response is to be generated. A preferred method is to clone out the sequence of the variable regions of the T cell receptor subunits. Then the variable sequences are linked to the sequence of the T cell receptor gene constant regions from the organism in which the immune response is to be generated. The hybrid T cell receptor thus has the desired antigen specificity, but originates from the same organism as the target cells.

The polynucleotide sequence of an antigen specific T cell receptor can be determined or generated by any technique known in the art. One technique available for obtaining the polynucleotide sequence of a T cell receptor is to isolate T cells that bind to a specific antigen and to determine the sequence of the T cell receptor (T cell receptor) encoded by that isolated clone. Such methods are well known in the art.

In one embodiment, a T cell receptor that recognizes an antigen of interest is identified by immunizing a humanized mouse that expresses certain human HLA allele(s) with the antigen of interest. T cell clones are generated that respond to the tumor antigen, which are restricted by the expressed human HLA allele(s). T cell receptors are then cloned from these T cell clones. A polynucleotide encoding a T cell receptor that recognizes the antigen of interest is identified and transferred into target cells as described below. The target cells are then transferred into the host in which an immune response to the antigen is desired, such as a patient suffering from or at risk of a disease or disorder with which the antigen is associated.

In another embodiment, a T cell receptor library of polynucleotides encoding T cell receptors with desired properties (e.g. high antigen responsiveness and/or the ability to collaborate with each other) is established from T cell clones. The T cell receptors may be whole cloned T cell receptors or hybrid T cell receptors as described above. The T cell receptor library is delivered into target cells, one T cell receptor per fraction, to generate antigen-specific T cells. This can be accomplished, for example, using the techniques described for a single gene (not a library) by Stanislawski, 2001, “Circumventing tolerance to a human MDM2-derived tumor antigen by T cell receptor gene transfer.” Nature Immunol. 2, 962-70.

Transformation of Target Cells

Once a polynucleotide encoding the desired T cell receptor is identified, it is introduced into a target cell. Preferably, the T cell receptor is introduced into the target cell in one vector. For example, the α and β subunits can be introduced together as a single polynucleotide. The two polynucleotides may be separated in the vector. In a preferred embodiment a polynucleotide encoding the α subunit of the T cell receptor and a polynucleotide encoding the β subunit of the T cell receptor are separated by an internal ribosome entry site (IRES).

However, in other embodiments, the T cell receptor is introduced into the target cell in more than one vector. For example, polynucleotides encoding the α and β subunit can be introduced separately into the target cell, each in an appropriate vector, for example each as a separate retroviral particle.

In other embodiments one or more polynucleotides are introduced into the target cell in addition to the polynucleotide(s) encoding the T cell receptor. For example, a polynucleotide that encodes a marker, such as green fluorescent protein (GFP), can be included. Such a marker can be used to determine if cells have been successfully transfected. In other embodiments, a polynucleotide may be included that encodes a polypeptide that may be used as a “switch” to disable or destroy cells transfected with the T cell receptor in a heterogeneous population. Such a switch may be included for safety reasons. In one such embodiment, a thymidine kinase gene (TK) is introduced into the target cells with the T cell receptor, the expression of which renders a target cell susceptible to the action of the drug gancyclovir.

In a preferred embodiment, one or more vectors are used to introduce the desired polynucleotides into the target cell. The vectors comprise the polynucleotide sequences encoding the T cell receptor and/or their complements, optionally associated with one or more regulatory elements that direct the expression of the coding sequences. Eukaryotic cell expression vectors are well known in the art and are available from a number of commercial sources. The choice of vector and/or expression control sequences to which the antigen-specific polynucleotide sequence is operably linked depends directly, as is well known in the art, on the functional properties desired, e.g., protein expression, and the target cell to be transformed. A preferred vector contemplated by the present invention is capable of directing the insertion of the T cell receptor polynucleotide into the host chromosome and the expression of the T cell receptor.

Expression control elements that may be used for regulating the expression of the T cell receptor are known in the art and include, but are not limited to, inducible promoters, constitutive promoters, secretion signals, enhancers and other regulatory elements.

In one embodiment, a vector comprising a T cell receptor polynucleotide will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

The vector may include a gene for a selectable marker that is effective in a eukaryotic cell, such as a drug resistance selection marker. This gene encodes a factor necessary for the survival or growth of transformed host cells grown in a selective culture medium. Host cells not transformed with the vector containing the selection gene will not survive in the culture medium. Typical selection genes encode proteins that confer resistance to antibiotics or other toxins, e.g., ampicillin, neomycin, methotrexate, or tetracycline, complement auxotrophic deficiencies, or supply critical nutrients withheld from the media. The selectable marker can optionally be present on a separate plasmid and introduced by co-transfection.

Vectors will usually contain a promoter that is recognized by the target cell and that is operably linked to the antigen-specific polynucleotide. A promoter is an expression control element formed by a DNA sequence that permits binding of RNA polymerase and transcription to occur. Promoters are untranslated sequences that are located upstream (5′) to the start codon of a structural gene (generally within about 100 to 1000 bp) and control the transcription and translation of the antigen-specific polynucleotide sequence to which they are operably linked. Promoters may be inducible or constitutive. Inducible promoters initiate increased levels of transcription from DNA under their control in response to some change in culture conditions, such as a change in temperature.

One of skill in the art will be able to select an appropriate promoter based on the specific circumstances. Many different promoters are well known in the art, as are methods for operably linking the promoter to the antigen-specific polynucleotide. Both native promoter sequences and many heterologous promoters may be used to direct expression of the antigen-specific polypeptide. However, heterologous promoters are preferred, as they generally permit greater transcription and higher yields of the desired protein as compared to the native promoter.

The promoter may be obtained, for example, from the genomes of viruses such as polyoma virus, fowlpox virus, adenovirus, bovine papilloma virus, avian sarcoma virus, cytomegalovirus, a retrovirus, hepatitis-B virus and Simian Virus 40 (SV40). The promoter may also be, for example, a heterologous mammalian promoter, e.g., the actin promoter or an immunoglobulin promoter, a heat-shock promoter, or the promoter normally associated with the native sequence, provided such promoters are compatible with the target cell. In one embodiment, the promoter is the naturally occurring viral promoter in a viral expression system.

Transcription may be increased by inserting an enhancer sequence into the vector. Enhancers are typically cis-acting elements of DNA, usually about 10 to 300 bp in length, that act on a promoter to increase its transcription. Many enhancer sequences are now known from mammalian genes (globin, elastase, albumin, α-fetoprotein, and insulin). Preferably an enhancer from a eukaryotic cell virus will be used. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the antigen-specific polynucleotide sequence, but is preferably located at a site 5′ from the promoter.

Expression vectors used in target cells will also contain sequences necessary for the termination of transcription and for stabilizing the mRNA. These sequences are often found in the 5′ and, occasionally 3′, untranslated regions of eukaryotic or viral DNAs or cDNAs and are well known in the art.

Plasmid vectors containing one or more of the components described above are readily constructed using standard techniques well known in the art.

For analysis to confirm correct sequences in plasmids constructed, the plasmid may be replicated in E. coli, purified, and analyzed by restriction endonuclease digestion, and/or sequenced by conventionai methods.

Vectors that provide for transient expression in mammalian cells of an antigen-specific polynucleotide may also be used. Transient expression involves the use of an expression vector that is able to replicate efficiently in a host cell, such that the host cell accumulates many copies of the expression vector and, in turn, synthesizes high levels of a the polypeptide encoded by the antigen-specific polynucleotide in the expression vector. Sambrook et al., supra, pp. 16.17-16.22.

Other vectors and methods suitable for adaptation to the expression of antigen-specific polypeptides are well known in the art and are readily adapted to the specific circumstances.

Using the teachings provided herein, one of skill in the art will recognize that the efficacy of a particular delivery system can be tested by transforming primary bone marrow cells with a vector comprising a gene encoding a reporter protein and measuring the expression using a suitable technique, for example, measuring fluorescence from a green fluorescent protein conjugate. Suitable reporter genes are well known in the art.

Transformation of appropriate cells with vectors of the present invention is accomplished by well-known methods, and the method to be used is not limited in any way. A number of non-viral delivery systems are known in the art, including for example, electroporation lipid-based delivery systems including liposomes, delivery of “naked” DNA, and delivery using polycyclodextrin compounds, such as those described in Schatzlein AG. 2001. Non-Viral Vectors in Cancer Gene Therapy: Principles and Progresses. Anticancer Drugs. Cationic lipid or salt treatment methods are typically employed, see, for example, Graham et al. Virol. 52:456, (1973); Wigler et al. Proc. Natl. Acad. Sci. USA 76:1373-76, (1979). The calcium phosphate precipitation method is preferred. However, other methods for introducing the vector into cells may also be used, including nuclear microinjection and bacterial protoplast fusion.

Preferred vectors for use in the methods of the present invention are viral vectors. There are a large number of available viral vectors that are suitable for use with the invention, including those identified for human gene therapy applications, such as those described in Pfeifer A, Verma I M. 2001. Gene Therapy: promises and problems. Annu. Rev. Genomics Hum. Genet. 2:177-211. Suitable viral vectors include vectors based on RNA viruses, such as retrovirus-derived vectors, e.g., Moloney murine leukemia virus (MLV)-derived vectors, and include more complex retrovirus-derived vectors, e.g., Lentivirus-derived vectors. Human Immunodeficiency virus (HIV-1)-derived vectors belong to this category. Other examples include lentivirus vectors derived from HIV-2, feline immunodeficiency virus (FIV), equine infectious anemia virus, simian immunodeficiency virus (SIV) and maedi/visna virus.

In one embodiment, a modified retrovirus, more preferably a modified lentivirus, is used to deliver the specific polynucleotide encoding the T cell receptor to the target cell. The polynucleotide and any associated genetic elements are thus integrated into the genome of the host cell as a provirus.

The modified retrovirus is preferably produced in a packaging cell from a viral vector that comprises the sequences necessary for production of the virus as well as the T cell receptor-encoding polynucleotide. The viral vector may also comprise genetic elements that facilitate expression of the antigen-specific polypeptide, such as promoter and enhancer sequences as discussed above. In order to prevent replication in the target cell, endogenous viral genes required for replication may be removed.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)), Coffin et al. (Retroviruses. Cold Spring Harbor Laboratory Press, N.Y. (1997)) and “RNA Viruses: A Practical Approach” (Alan J. Cann, Ed., Oxford University Press, (2000)).

The viral vector may incorporate sequences from the genome of any known organism. The sequences may be incorporated in their native form or may be modified in any way. For example, the sequences may comprise insertions, deletions or substitutions. In a preferred embodiment the viral vector comprises an intact retroviral 5′ LTR and a self-inactivating 3′ LTR.

Any method known in the art may be used to produce infectious retroviral particles whose genome comprises an RNA copy of the viral vector. To this end, the viral vector is preferably introduced into a packaging cell line that packages viral genomic RNA based on the viral vector into viral particles with a desired target cell specificity. The packaging cell line provides the viral proteins that are required in trans for the packaging of the viral genomic RNA into viral particles. The packaging cell line may be any cell line that is capable of expressing retroviral proteins. Preferred packaging ceil lines include 293 (ATCC CCL X), HeLa (ATCC CCL 2), D17 (ATCC CCL 183), MDCK (ATCC CCL 34), BHK (ATCC CCL-10) and Cf2Th (ATCC CRL 1430).

The packaging cell line may stably express the necessary viral proteins. Such a packaging cell line is described, for example, in U.S. Pat. No. 6,218,181. Alternatively a packaging cell line may be transiently transfected with plasmids comprising nucleic acid that encodes the necessary viral proteins.

Viral particles are collected and allowed to infect the target cell. Target cell specificity may be improved by pseudotyping the virus. Methods for pseudotyping are well known in the art.

In one embodiment, the recombinant retrovirus used to deliver the antigen-specific polypeptide is a modified lentivirus. As lentiviruses are able to infect both dividing and non-dividing cells, in this embodiment it is not necessary to stimulate the target cells to divide.

In another embodiment the vector is based on the murine stem cell virus (MSCV). The MSCV vector provides long-term stable expression in target cells, particularly henatopoietic precursor cells and their differentiated progeny.

A DNA viral vector may be used, including, for example adenovirus-based vectors and adeno-associated virus (AAV)-based vectors. Likewise, retroviral-adenoviral vectors also can be used with the methods of the invention.

Other vectors also can be used for polynucleotide delivery including vectors derived from herpes simplex viruses (HSVs), including amplicon vectors, replication-defective HSV and attenuated HSV. [Krisky D M, Marconi P C, Oligino T J, Rouse R J. Fink D J, et al. 1998. Development of herpes simplex virus replication-defective multigene vectors for combination gene therapy applications. Gene Ther. 5: 1517-30]

Other vectors that have recently been developed for gene therapy uses can also be used with the methods of the invention. Such vectors include those derived from baculoviruses and alpha-viruses. [Jolly D J. 1999. Emerging viral vectors. pp 209-40 in Friedmann T, ed. 1999. The development of human gene therapy. New York: Cold Spring Harbor Lab].

These and other vectors can also be used in combination to introduce one or more T cell receptor-encoding polynucleotides according to the invention.

Recombinant virus produced from the viral vector may be delivered to the target cells in any way that allows the virus to infect the cells. Preferably the virus is allowed to contact the cell membrane, such as by incubating the cells in medium that comprises the virus. Preferably, spin-infectin is used, as is well known in the art.

Target cells include both germline cells and cell lines and somatic cells and cell lines. Target cells can be stem cells derived from either origin. When the target cells are germline cells, the target cells are preferably selected from the group consisting of single-cell embryos and embryonic stem cells (ES). When the target cells are somatic cells, the cells include, for example, mature lymphocytes as well as hematopoietic stem cells.

A target cell may be a stem cell or stem cell line, including without limitation heterogeneous populations of cells that contain stem cells. Preferably, the target cells are hematopoietic stem cells. More preferably the target cells are primary bone marrow cells. Target cells can be derived from any mammalian organism including without limitation, humans, pigs, cows, horses, sheep, goats, rats, mice, rabbits, dogs, cats and guinea pigs. Target cells may be obtained by any method known in the art. Preferably, target cells are obtained from a mammal in need of treatment. Target cells may be transfected either in vivo or in vitro. Preferably, target cells are maintained in culture and are contacted transfected in vitro. Methods for culturing cells are well known in the art.

Depending on the vector that is to be used, target cell division may be required for transformation. Target cells can be stimulated to divide in vitro by any method known in the art. For example, hematopoietic stem cells can be cultured in the presence of one or more growth factors, such as IL-3, IL-6 and/or stem cell factor (SCF).

Control of Cell Fate, Using T Cell Receptors to Create CD4 or CD8 Cells

It has been found that the original source of the T cell receptor can direct the development of immune cells in the host. That is, if a T cell receptor is obtained from a helper T cell, target cells transfected with the T cell receptor will develop into helper T cells in the host. On the other hand, if the T cell receptor is obtained from a cytotoxic T cell, target cells transfected with the T cell receptor will develop into cytotoxic T cells in the host. In other words, if the T cell receptor is one that is found in helper T cells, then expression of the T cell receptor in stem cells will result in the development of T helper cells in the host. Similarly, if the T cell receptor is one that is found in cytotoxic T cells, then expression of the T cell receptor in stem cells will result in the development of cytotoxic immune cells in the host.

This feature can be used to direct the immune response generated in a patient. For example, in some situations the generation of helper T cells is desirable. Thus, a T cell receptor specific for the disease associated antigen of interest is identified from helper T cells. Stem cells are transfected with this type of T cell receptor and the cells are then transferred into the patient, where they will then develop into helper T cells. On the other hand, if it is desirable to produce cytotoxic T cells in a patient that are specific for the disease associated antigen, a T cell receptor that is specific for that antigen is used to transfect target cells.

In other embodiments, both cytotoxic T cells and helper T cells are generated in the host. In these embodiments, a first T cell receptor is identified from a helper T cell and a second T cell receptor is identified from a cytotoxic T cell. These two T cell receptors are then separately introduced into target cells which are then transferred back to the patient where they develop into helper T cells and cytotoxic T cells respectively. In this way, both arms of the T cell immunity are generated in a patient.

T cells that are specific to more than one disease-associated antigen or epitope may also be generated in a patient. That is, if more than antigen (or more than one epitope on the same antigen) can be identified that is associated with the disease or disorder from which a patient is suffering, T cells can be separately generated for each of those antigens. Thus, in one embodiment, a T cell receptor is identified that is specific for a first disease-associated antigen and a second T cell receptor is identified that is specific for a second disease-associated antigen. These T cell receptors are separately transfected into target (cells which are then transferred into the patient. Two distinct populations of T cells then develop in the patient each of which is able to generate an immune response to its specific disease-associated antigen. The number of antigens or epitopes that can be targeted in this way is not limited. In addition, for each antigen it is possible to identify a T cell receptor from cytotoxic T cells or helper T cells as discussed above. Thus, one or both arms of the T cell immunity (cytotoxic T cells and helper T cells) can be generated in the patient for each of the multiple disease-associated antigens that have been identified.

Enhancing the Immune Response with Immunization

In preferred embodiments, patients in which T cells have been generated are immunized with the antigen or antigens for which the generated T cells have specificity. Methods of immunization are well known in the art and are described, for example, in Schuler (2003) Cancer Immunity, 3:23.

Immunization is preferably performed at a time point following introduction of transfected target cells to the patient, more preferably after allowing sufficient time for T cells to develop in the patient. For example, a patient may be immunized at least one, two, three, four, five or more days following immunization. Optionally, immunization may be contemporaneous with introduction of transfected target cells to the patient.

Immunization may be carried out a single time, or repeated as desired. For example, immunizations may be repeated at regular intervals, to prevent disease progression. Thus, in some embodiments repeated immunizations are utilized to control a disease for an extended period of time or to completely eliminate a disease. In particular embodiments, multiple immunizations are utilized to prevent tumor recurrence. The appropriate interval between immunizations may be determined by the skilled practitioner based on specific circumstances. For example, the immunization may be repeated weekly, monthly, bimonthly, quarterly, biannually or annually.

In preferred embodiments, dendritic cells are used to immunize a patient. Immunization with dendritic cells is described for example in Fong et al. (2001) Journal of Immunology 166:4254-4259 and Steinman et al. International Journal of Cancer 1994 459-473 (2001), which are incorporated herein by reference. Dendritic cells (DCs) are antigen-presenting cells that are able to induce specific T cell immunity. Briefly, dendritic cells can be harvested from the patient or from a donor. The dendritic cells can then be exposed in vitro to the disease-associated antigen for which T cells are to be generated in the patient. Dendritic cells loaded with the antigen are then injected back into the patient. Immunization is preferably preferred after the T cells have generated in the patient. Immunization may be repeated multiple times if desired. Methods for harvesting, expanding, and administering dendritic cells are well known in the art, for example, as described in Fong et al., supra.

In other embodiments the antigen is administered to the patient directly. The antigen may be associated with a carrier or excipients as is known in the art.

Adoptive Immunotherapy

In other embodiments, the methods of the present invention can be used for adoptive immunotherapy in a patient. As described above, an antigen against which an immune response is desired is identified. A T cell receptor that is specific for the antigen is then identified and a polynucleotide encoding the T cell receptor is obtained. Target cells, preferably hematopoietic stem cells, more preferably primary bone marrow cells are obtained from the patient and transfected with a polynucleotide that encodes the T cell receptor. The target cells are then transferred back into the patient.

After sufficient time to allow the target cells to develop into mature immune cells, T lymphocytes are harvested from the patient. This may be done by any method known in the art. Preferably, lymphocytes are isolated from a heterogeneous population of cells obtained from peripheral blood. They may be isolated, for example, by gradient centrifugation, fluorescence activated cell sorting (FACS), panning on monoclonal antibody coated plates or magnetic separation techniques. Antigen specific clones are then isolated by stimulating cells, for example with antigen presenting cells or anti-CD3 monoclonal antibody, and subsequent cloning by limited dilution or other technique known in the art. Clones that are specific for the antigen of interest are identified, expanded and transferred back into the patient, such as by infusion into the peripheral blood.

### EXAMPLES

A desired anti-tumor specificity was imparted to the T cell repertoire of a host by delivering tumor-specific T cell receptor genes into HSCs. This was followed by adaptive transfer to generate a continuous stream of anti-tumor T cells.

The E.G7 mouse tumor model was used to test the disclosed methods of immunotherapy. E.G7 is a mouse thymoma cell line, which was generated by engineering the parent cell line EL.4 to express the chicken OVA gene (Moore et al. Cell 54:777 (1998)). OVA is the well characterized target antigen for both OT1, a well-characterized CD8 TCR, and OT2, a well known CD4 TCR.

Previous studies showed that the OVA₂₅₇₋₂₆₄ peptide displayed on the surface of E.G7 cells, in the context of MHC class 1 molecule H-2 K^{b}, can be recognized by the OT1 T cell receptor (Hogquist et al. Cell 76:17 (1994)) at a density (100H-2k^{b}/OVA₂₃₇₋₂₆₄ per cell) similar to the density of tumor antigens on authentic tumor cells (Rotzschke et al. Eur. J. Immunol. 21:2891 (1991)). Thus, the E.G7 tumor cell-OT1 T cell system has been widely used to study cytotoxic T cell (CTL) mediated anti-tumor immune responses (Shrikant et al. Immunity 11:482 (1999) and Helmich et al. J. Immunol. 166:6500 (2001)).

Further, the natural processed OVA₃₂₃₋₃₃₉ peptide presented by MHC class II molecule I-A^{b} is recognized by the CD4 T cell receptor OT2 (Barnden et al., 1998), providing an opportunity to study the anti-tumor CD4 T cell immune response as well.

Experimental Methods

The following experimental methods were used in Examples 1-8 below.

Mice

C57BL/6J(B6) female mice were purchased from Charles River Breeding Laboratories, and RAG1 deficient female mice in the B6 background were purchased from The Jackson Laboratory. OT2 T cell receptor transgenic mice in B6 background were also purchased from The Jackson Laboratory and then bred into RAG1 deficient background to generate OT2/RAG1 T cell receptor transgenic mice. All mice were housed in the California Institute of Technology animal facility in accordance with institute regulations.

MOT1 and MOT 0.2 Retrovirus

The MOT1 and MOT2 construct was generated from the MIG retrovirus by replacing GFP with the OT1 or OT2 T cell receptor beta chain cDNA and inserting the OT1 or OT2 T cell receptor α chain cDNA in the vector upstream of the IRES (Yang L. et al. 2002. Proc. Natl. Acad. Sci. USA 99:6204-6209. The MIG retroviral expression vector is described in Van Parijs L. et. al, 1999, Immunity, 11:281-288. Retroviruses were made in HEK293.T cells and harvested 36-48 hours after transfection.

Peptides

OVA₂₅₇₋₂₆₄ peptide (designated as OVAp1) recognized by the OT1 T cell receptor and OVA₃₂₃₋₃₃₉ peptide (designated as OVAp2) recognized by the OT2 T cell receptor were all synthesized at the Cal Tech Biopolymer Synthesis Center.

Primary T Cell Infection and Stimulation

Spleen cells were harvested from B6 female mice of six to eight weeks age and activated in vitro with 0.5 μg/ml anti-CD3 and 0.5 μg/ml anti-CD28 Abs (both from Pharmingen). On day 2 of culture, cells were spin-infected with MOT1 or MOT2 retroviruses in the presence of 110 g/ml polybrene for 90 min at 2,500 rpm at 30° C. On day 3, cells were collected for analysis. Some aliquots of the collected cell were used to assay for the expression of OT1 or OT2 T cell receptors by flow cytometry. The remaining aliquots were allowed to rest overnight with 10 ng/ml RMIL-2 (Biosource International, Camarillo, Calif.). The next day, the rested cells were tested for responsiveness to antigen stimulation. The cells infected with MOT1 retrovirus were stimulated with OVAp1 at 0 to 1 μg/ml in the presence of APCs (spleen cells of B6 female mice). The cells infected with MOT2 retrovirus were stimulated with OVAp2 at 0 to 10 μg/ml in the presence of APCs (spleen cells of B6 female mice). On day 3 of stimulation, cell cultures supernatants were collected and analyzed for IFN-γ production using ELISA.

Hematopoietic Stem Cells (HSCs) Isolation Infection and Transfer

B6 female mice or RAG1^{-/-} female mice (6-8 weeks old) were treated with 250 μg/g of body weight of 5-fluorouracil (Sigma). Five days later, bone marrow (BM) cells enriched with HSCs were harvested and cultured for 4 days in RPMI containing 10% FBS with 20 ng/ml rmIL-3, 50 ng/ml rmIL-6 and 50 ng/ml rmSCF (all from Biosource International, Camarillo, Calif.). On day 2 and 3, the cells were spin infected with MOT1 or MOT2 retroviruses supplemented with 8 μg/ml polybrene for 90 min at 2,500 rpm, 30° C. On day 4 of culture, BM cells were collected and transferred by tail vein injection into B6 female hosts or RAG1^{-/-} female hosts that had received 1200 rads or 360 rads whole-body radiation. Each host received 2-3×10⁶ infected BM cells. BM recipient mice were maintained on a mixed antibiotic sulfinethoxazole and trimethoprim oral suspension (Hi-Tech Pharmacal, Amityville, N.Y.) in a sterile environment for 6-8 weeks until analysis or usage for further experiments.

In Vitro T Cell Stimulation and Functional Assays

For antigen dose-response experiments, spleen cells from BM recipient mice were harvested and cultured at 2×10⁵ cells/well in T cell culture medium containing OVAp1 at 0-1 μg/ml or OVAp2 at 0-10 μg/ml. Three days later, culture supernatants were collected and assayed for IL-2, IL-4 or IFN-γ production by ELISA, and proliferation was assessed by [³H]thymidine incorporation.

For time course response, cells were stimulated with 0.1 μg/ml OVAp1 or 1 μg/ml OVAp2, and the culture supernatants were collected and assayed for IL-2, IL-4 or IFN-γ production by ELISA on day 1.5, day 2.5 and day 3.5. In cytokine proliferation response, cells were cultured with 10 ng/ml rmIL-2, 10 ng/ml IL-4, or 10 ng/ml rmIL-15 (BioSource International, Camarillo, Calif.) for 4 days in the absence of antigen and proliferation was assessed by [³H]thymidine incorporation.

Antibodies and FACS Analysis

Fluorochrome-conjugated antibodies specific for mouse CD4, CD8, CD25, (CD69, CD62L, CD44, T cell receptor Vα2, and T cell receptor Vβ5.1, 5.2 were purchased from BD Pharmingen (San Diego, Calif.). Surface staining was performed by blocking with anti-CD 16/CD32 (mouse Fc receptor, BD Pharmingen, San Diego, Calif.) followed by staining with fluorochrome-conjugated antibodies. Intracellular staining of T cell receptor was done using the Cytofix/Cytoperm™ Kit from BD Pharmingen (San Diego, Calif.). Analyses were performed on a FACScan flow cytometer.

T Cell Memory Study

Spleen and lymph node cells from BM recipient mice (B6/MOT1) were harvested and stimulated with 0.1 μg/ml OVAp1 or 1 μg/ml OVAp2 for 3 days in vitro, respectively. The cells were then collected and transferred into RAG1^{-/-} hosts by tail vein injection. Each host received 20-30×10⁶ cells (>10% were activated OT1 or OT2 T cells). Sixteen weeks later, spleen cells were harvested from the hosts and analyzed for the presence of long-lived OT1 or OT2 T cells. Memory phenotype of the OT1 or OT2 T cells was studied by FACS Memory function was studied by antigen dosage response, antigen time-course response and cytokine proliferation response of the OT1 or OT2 T cells. For antigen dosage response, cells were stimulated with 0-1 μg/ml OVAp1 or 0-10 μg/ml OVAp2 for 3 days, and the culture supernatants were collected and assayed for IL-2, IL-4 or IFN-γ production by ELISA. Proliferation was assessed by [H]thymidine incorporation. For an antigen time-course response, cells were stimulated with 0.1 μg/ml OVAp1 or 1 μg/ml OVAp2, and the culture supernatants were collected and assayed for IL-2, IL-4 or IFN-γ production by ELISA on day 1.5, day 2.5 and day 3.5. In cytokine proliferation response, cells were cultured with 10 ng/ml rmIL-2, 10 ng/ml rmIL-4 or 10 ng/ml rmIL-15 (all from BioSource International, Camarillo, Calif.) for 4 days in the absence of antigen, and proliferation was assessed by [³H]thymidine incorporation.

Tumor Challenge of Mice

The tumor cell lines EL.4 (C57BL/6, H-2b, thymoma) and E.G7 (EL.4 cells transfected with the chicken OVA cDNA) (Moore et al., 1988) were used for tumor challenge. 5×10⁶ EL.4 or E.G7 cells were injected subcutaneously into the left flank of the mice. Tumor size was measured every other day using fine calipers (Manostat Corporation, Switzerland), and is shown as the product of the two largest perpendicular diameters a×b (mm²). Mice were euthanized when the tumors reached 400 mm².

Dendritic Cell Generation, Antigen Pulsing and Mouse Immunization

Dendritic cells (DC) were generated from bone marrow cultures as described by Lutz M B et al. (Lutz et al., 1999. J. Immunol. Methods 223:77-92), with some minor modifications. Briefly, bone marrow cells were harvested from B6 female mice (6-8 weeks old) and cultured in 10 cm diameter petri dishes at 2×10⁶ cells/dish in 10 ml RIO medium (RPMI-1640 supplemented with 100 U/ml Penicillin, 100 μg/ml Streptomycine, 2 mM L-glutamin, 50 μM 2-mercaptoethanol and 10% FBS) containing 1:30 J558L culture supernatant. J558L is a cell line transfected with the murine GM-CSF gene (Zal et al., 1994) and its culture supernatant was used as the source of GM-CSF. On day 3 another 10 ml R10 medium containing 1:30 J558L culture supernatant was added to each dish. On day 6 and day 8, half of the culture supernatant was collected and centrifuged, and the cell pellet was resuspended in 10 ml fresh RIO medium containing 1:30 J558L culture supernatant and added back into the original culture dishes. On day 9, non-adherent cells were collected and plated into new 10 cm diameter petri dishes at 4-6×10⁶ cells/dish in 10 ml RIO medium containing 1:60 J558L culture supernatant and LPS (1 μg/ml; Sigma) to mature DCs. On day 10, non-adherent cells (usually >80% are mature DCs) were collected and washed once with IMDM/50 mM 2-mercaptoethanol and resuspended in 0.8 ml of the same medium containing 100 μg OVAp1 (or 100 μg OVAp1 plus 100 μg OVAp2). The cells were then incubated at 37° C. for 3 hours with gentle shaking every 30 min. Three hours later, the OVAp1 or OVAp1 plus 2 loaded DCs were washed twice with PBS and used to immunize mice by tail vein injection, Each mouse received about 0.5×10⁶ OVAp loaded DCs.

### Example 1

### Functional Expression of CD8 and CD4 T Cell Receptors (T Cell Receptors)

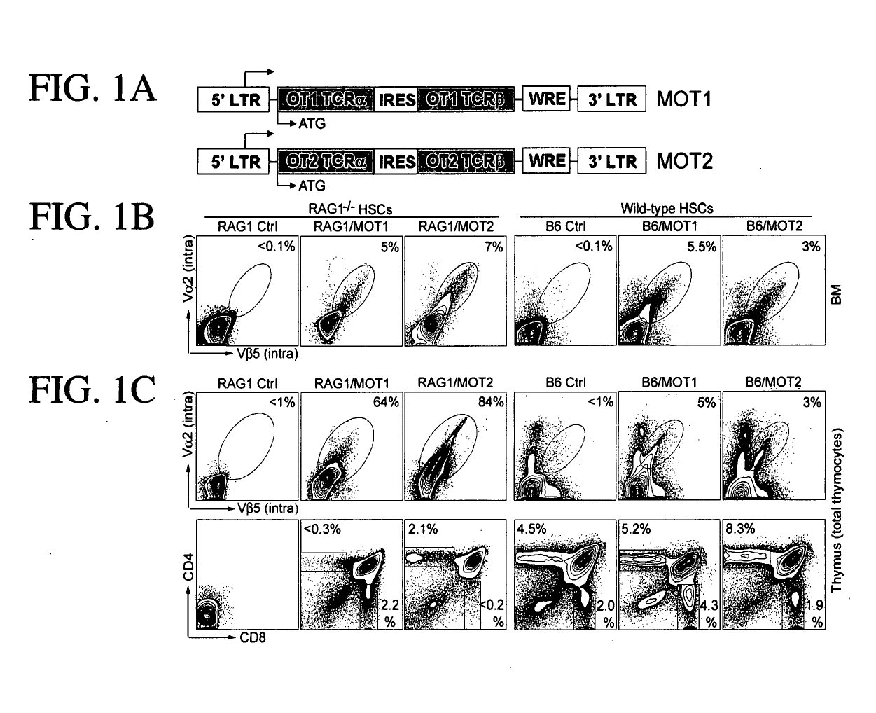

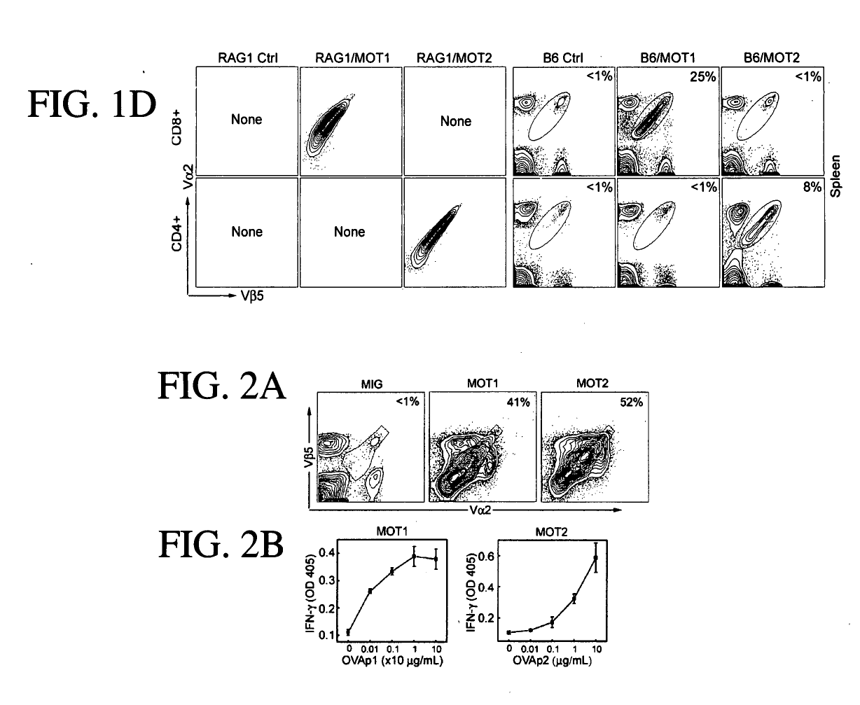

The OT1 T cell receptor recognizes chicken OVAp257.264 (denoted as OVAp1 herein) and the OT2 T cell receptor recognizes chicken OVAp₃₂₃₋₃₃₉ (denoted as OVAp2 herein). Both T cell receptors were cloned from B6 mice (Barnden et al. 1998; Kelly et al. 1993). The cDNAs encoding the OT1 or OT2 T cell receptor α and β chains were inserted into a retroviral vector based on MSCV (mouse stem cell virus) under the control of the viral LTR promoter. The resulting constructs were designated as MOT1 and MOT2, respectively (FIG. 1A). To achieve co-expression of the T cell receptor α and β chains, the two cDNAs were linked with an IRES element.

When MOT1 retroviruses were used to infect activated mouse T cells. OT1 T cell receptors were observed on the cell surface in 41% of the cells, as shown in FIG. 2A, and the infected cells were able to respond to OVAp stimulation to produce the effector cytokine IFN-γ (FIG. 2B). Similarly, when MOT2 retroviruses were used to infect in vitro activated mouse peripheral T cells, 52% of the cells were found to express OT2 T cell receptors (FIG. 2A). These cells were able to respond to OVAp2 stimulation to produce IFN-γ (FIG. 2B). These results show that the viral constructs can efficiently mediate functional expression of CD4 and CD8 T cell receptors.

### Example 2

### Generation of Monospecific CD8 and CD4 T Cells by Retrovirus-Mediated Expression of CD8 and CD4 T Cell Receptors

This example demonstrates that T cell fate is controlled by the nature of the transgenic r cell receptor gene; thus, one can select the type of immune cell to be created based on the origin of the T cell receptor to be expressed. MOT1 and MOT2 constructs were used to generate OT1 CD8 cytotoxic cells and OT2 CD4 T helper cells from wild type hematopoietic stem cells (HSCs) in vivo.

Antigen-specific T cells were generated in RAG1 deficient (RAG^{-/-}) mice and wild type mice (B6). B6 wild-type and RAG1^{-/-} HSCs were infected with retrovirus generated from a single retroviral vector comprising cDNAs encoding the α and β chains of OT1 (MOT1) or OT2 T cell receptor (MOT2) linked by an internal ribosome entry site (IRES).

RAG1^{-/-} and B6 mice were treated with 5-FU to enrich the HSCs in bone marrow (BM). Five days later, BM cells were harvested and cultured in vitro and the cells were infected with MOT1 or MOT2 retroviruses. The transduced HSCs were collected and transferred separately into irradiated RAG1⁻⁻ or B6 recipient mice. The resulting mice were designated as RAG1/MOT1, RAG1/MOT2, B6/MOT1 and B6/MOT2 respectively. The recipient mice were allowed to reconstitute their immune system for at least 6 weeks.

Seven weeks after adoptive transfer, the recipients were analyzed. From about 3% to about 7% of cells in BM expressed the OT1 or OT2 transgenic T cell receptors, respectively (FIG. 1B). Analysis showed the presence of long-term HSCs, as identified by the surface markers c-Kit and Scal-1.

Study of the recipients 6 to 8 months after adoptive transfer showed the persistence of the transduced HSCs. In addition, these cells persisted through secondary transfer of the recipient BM cells. Taken together, the results indicate that T cell receptor genes can be transferred into HSCs that maintain the stem cell features of longevity and self-renewal. In addition, expression of the T cell receptors was not silenced over time. Thus, this method of producing modified T cells is sufficiently robust to ensure the maintenance of an introduced T cell population for the lifetime of the host.

T cell development in the thymus was also analyzed. As shown in Figure 1C, the majority of the thymocytes expressed the OT1 or OT2 T cell receptor transgenes (64% in RAG1/MOT1 and 84% in RAG1/MOT2), indicating that they developed from the virally transduced HSCs. Further study of the distribution of T cell development markers CD4 and CD8 on thymocytes of the RAG1/MOT1 mice shows a typical pattern for CD8 T cell development. Due to the lack of endogenous T cell receptor rearrangement, T cells do not develop naturally in RAG1^{-/-} mice. In the thymus, the natural RAG1^{-/-} thymocytes stay at the double negative (DN) stage (FIG. 1C, lower left). It was found that in RAG1/MOT1 mice, T cell development was rescued and the RAG1^{-/-} thymocytes advanced to double positive (DP) stage, followed by advancement to the CD8 single positive (SP) stage (FIG. 1C, lower middle). Similarly, thymocytes of RAG1/MOT2 mice showed a rescued CD4 T cell development (FIG. 1C, lower right).

No leakage into the CD4 single positive T cell compartment was observed in RAG1/MOT1 mice. Similarly, no leakage into the CD8 single positive compartment was observed in RAG1/MOT2 mice. Peripheral lymph organs (spleen and lymph notes) were analyzed for the presence of mature T cells. In RAG1/MOT1 mice, CD8 T cells were found to be uniformly expressing OT1 T cell receptors, and no CD4 T cells were detected (FIG. 1D, upper left). In contrast, CD4 T cells were found to be uniformly expressing OT2 T cell receptor, and no CD8 T cells were detected in RAG1/MOT2 mice (FIG. 1D, lower left).

In the thymi of B6/MOT1 and B6/MOT2 mice, thymocytes expressing transgenic TCRs were detected and the CD8 and CD4 SP T cell compartment augmented compared to wild type animals (FIG. 1C). Further analysis of peripheral T cells showed that in a representative experiment 25% of the total peripheral CD8 T cells in B6/MOT1 expressed OT1 TCR and 8% of the total peripheral T cells in B6/MOT2 mice expressed OT2 TCR, with no leakage into the other compartments (FIG. 1D). In numerous experiments, an average of about 20% of the total peripheral CD8 T cells in B6/MOT1 mice carried the OT1 TCR specificity and an average of about 8% of the total peripheral CD4 T cells carried the OT2 TCR specificity. The high percentage of antigen specific T cells remained constant for 8 months post-transfer.

These results indicate that retrovirus-mediated expression of T cell receptor cDNAs in wild type HSC can stably generate a significant population of T cells with the desired features and specificity. Moreover, the determination of CD4 or CD8 T cell fate is strictly controlled by the nature of the transgenic T cell receptor genes.

### Example 3

### Comparison of the Transgenic T Cell Receptor Expression and T cell Development in Mice Receiving Retrovirus-Transduced RAG1^{-/-} HSCs with Those in the Conventional T Cell Receptor Transgenic Mice

To evaluate the efficacy of the generation of antigen-specific T cells in vivo, a comparison was made to a conventional transgenic mouse. An OT2 r cell receptor transgenic mouse was previously made by inserting the cDNA encoding the OT2 α chain into a pES4 transgenic expression construct that contained the H-2 K^{b} promoter, the IgH chain enhancer and the polyadenylation signal sequence of the human β-globin gene. The OT2 β chain gene was inserted into a genomic-based construct (Barnden et al., 1998. Immunol. Cell Biol. 76:34-40).

A detailed comparison of OT2 T cell receptor expression and T cell development between the RAG1/MOT2 recipient mice described above and the commercially available OT2/RAG1 transgenic mice (the conventional OT2 T cell receptor transgenic mice that have bred into RAG1^{-/-} background, designated as OT2/RAG1 Tg) was performed. The RAG1 genetic deficiency does not support endogenous T cell receptor expression, thus providing a clean background for the study.

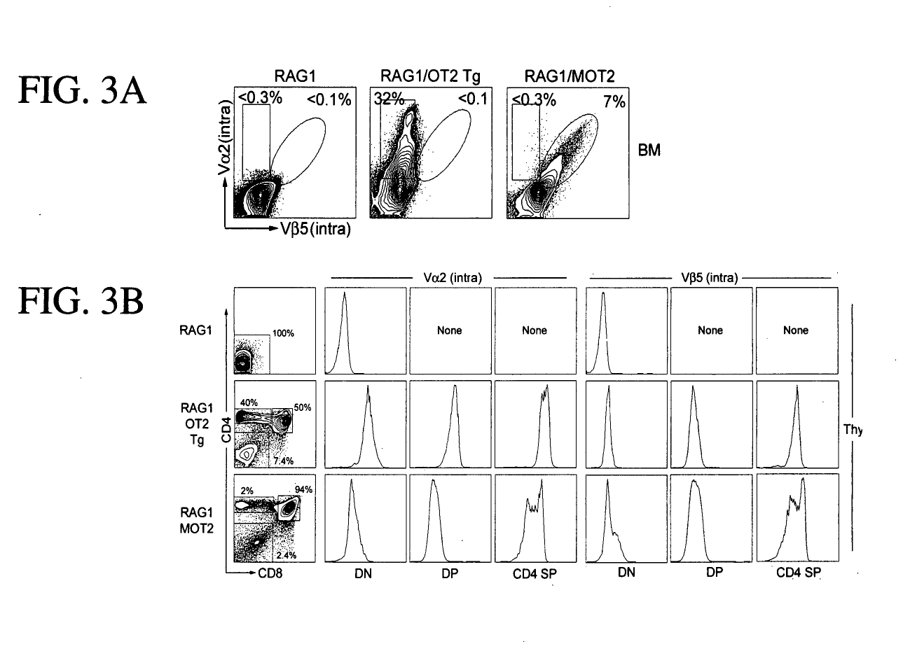

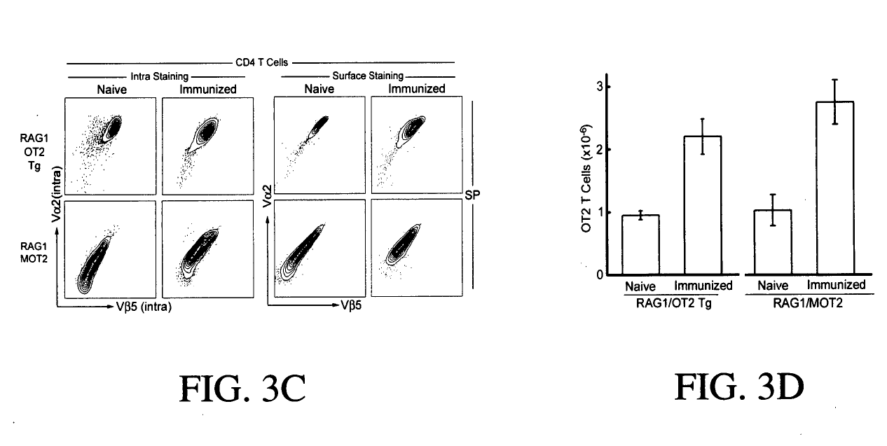

First, OT2 T cell receptor expression in BM was examined. T cell progenitors are derived from these cells. Since T cell receptors cannot display on the cell surface without associating with the CD3 proteins, which are only expressed in committed T lineage cells (Oettgen et al., 1986), intracellular staining was used to analyze T cell receptor expression in BM cells. In OT2/RAG1 Tg mice, a large portion (˜32%) of the BM cells expressed OT2 α chain (FIG. 3A, middle). No β chain expression was detected (FIG. 3A, middle). This observation is consistent with how the transgenes were constructed: the OT2 α chain is under control of H-2K^{b} promoter and IgH enhancer and the β chain is under control of natural T cell receptor promoter and enhancer (Barnden et al., 1998).

In the RAG1/MOT2 mice, 7% of the BM cells expressed OT2 genes and the α chains always co-localized with the β chains (FIG. 3A, right). This indicates that the MOT2 retroviruses effectively mediate co-expression of OT2 α and β cDNAs in hematopoietic cells.

The thymus was also analyzed. In RAG1^{-/-} control mice, thymocytes stopped at the DN stage due to the failure of rearrangement of endogenous T cell receptors (FIG. 3B, upper left). In OT2/RAG1 Tg mice, the development is rescued and the thymocytes show a typical CD4 T cell development pattern (FIG. 3B, middle left). Similarly, thymocytes in RAG1/MOT2 mice are rescued and develop CD4 SP T cells (FIG. 3B, lower left).

012 T cell receptor expression was followed in OT2/RAG Tg mice through the developmental stages (DN, DP and CD4 SP). The α chain was expressed constantly high through all the three stages; while the β chain expression started from DN stage, at a low level, up-regulated slightly in DP stage and reached a high level in CD4 SP stage (FIG. 3B). Interestingly, in RAG1/MOT2 mice, expression of both the OT2 α and β chains closely resembled the pattern of β chain expression in OT2/RAG1 Tg mice, which is under control of the natural T cell receptor promoter and enhancer (FIG. 3B, lower right).

When MOT2 retroviruses were used to infect HSCs, a large number of HSCs that expressed OT2 T cell receptor genes at a broad range were generated due to differences in viral copy numbers and the integration sites each HSC received. Heterogeneous T cell receptor expression was observed in BM cells (FIG. 3A, right) and DN thymocytes of RAG1/MOT2 mice (FIG. 3B, right). Subsequently, thymocytes expressing a slightly higher level of T cell receptors were allowed to enter the DP stage. A final selection of thymocytes expressing an even higher level of T cell receptors led to the generation of CD4 SP T cells (FIG. 3B, lower right). The CD4 SP thymocytes accounted for about 2% of the total thymocytes in RAG1/MOT1 mice, much less than the 42% in OT2/RAG1 Tg mice (FIG. 3B, left).

Finally, the presence of mature T cells in peripheral lymph organs was analyzed. As expected, only monospecific OT2 CD4 T cells and no CD8 T cells were observed in both OT2/RAG1 Tg mice and RAG1/MOT2 mice. Compared with the OT2 T cells in OT2/RAG1 Tg mice, the OT2 T cells in RAG1/MOT2 mice expressed T cell receptors in a broader range and with a lower average level, both at the level of protein expression as measured by intracellular staining, (FIG. 3C, left) and the surface display as measured by surface staining (FIG. 3C, right).

It is possible that lower expression of T cell receptor could impair the ability of T cells to respond to antigens. To address this concern, the antigen responsiveness of the OT2 T cells from RAG I/MOT2 mice was tested in vivo by immunizing the animals with OVAp2 peptide antigen. OT2/RAG1 Tg mice were included as a control. As shown in FIG. 3C, compared to the OT2 T cells in unchallenged mice, the OT2 T cells in immunized RAG1/MOT2 mice expressed T cell receptors above a certain level (judged by the intensity of intracellular T cell receptor staining, FIG. 3C), indicating that these cells responded to antigen stimulation and were preferentially expanded. On the contrary, no such change was observed in immunized OT2/RAG1 Tg mice (FIG. 3C). This result supports a quantitative signal threshold for T cell responsiveness, as reflected in FIG. 3C by the T cell receptor expression level.

In RAG1/MOT2 mice, the unresponsive cells expressing T cell receptors below the threshold accounted for less than 10% of the total OT2 T cells generated in RAG1/MOT2 mice (FIG. 3C). For the OT2 T cells above that threshold, the variation on T cell receptor expression level did not affect the ability of the T cells to respond, and no obvious expansion advantage was observed for OT2 T cells expressing higher level of T cell receptors (FIG. 3C). This observation is also confirmed by the in vitro T cell stimulation. In spite of the differences, comparison of the naïve OT2 T cell generation and the overall T cell expansion in response to antigen stimulation in vivo showed that the efficacy of the present methods in generating antigen specific T cells and the antigen-induced T cell response is at least comparable to that of the conventional T cell receptor transgenic technique (FIG. 3D).

In summary, during T cell development, the T cell receptor expression pattern mediated by the retroviral LTR promoter closely resembles the expression pattern produced by the natural T cell receptor promoter and enhancer in conventional transgenic mice. T cell development is normal in mice receiving retrovirus-transduced HSCs, and the disclosed methods are very efficient in generating functional antigen-specific T cells.

### Example 4

### Generation of Antigen-Specific CD8 or CD4 T Cells by Retrovirus-Mediated Expression of CD8 or CD4 T Cell Receptor in Wild-Type HSCs

This Example demonstrates that T cell receptor genes can be stably transferred into wild-type HSCs without affecting the stem cell features of longevity and self-renewal and thus provide the recipient of the HSCs with a lifelong source of hematopoietic cell progenitors with the desired genetic modifications and thus a lifelong source of the desired T cells.

A similar approach to that used with RAG1^{-/-} HSCs, as described above, was used to test wil-type HSCs. Wild-type B6 mice were treated with 5-FU to enrich the HSCs, and BM cells were harvested 5 days later. The cells were infected with either MOT1 or MOT2 retroviruses and transferred into irradiated B6 recipient mice (designated as B6/MOT1 or B6/MOT2). The recipients were allowed to reconstitute their immune system for at least 6 weeks.

Eight weeks after adoptive transfer, the B6/MOT1 and B6/MOT2 mice were analyzed. As in the RAG1/MOT1 and RAG1/MOT2 mice, BM cells expressing the transgenic OT1 and OT2 T cell receptors were observed in B6/MOT1 mice (5.5%, FIG. 1B) and B6/MOT2 mice (3%, FIG. 1B), respectively. Analysis of the stem cell markers c-Kit and Scal-1 indicated that these cells included long-term HSCs.

Study of the recipients 8 months after adoptive transfer demonstrated the persistence of the transduced HSCs. Furthermore, the transduced cells were observed to persist through secondary transfer. These results are consistent with the results obtained using RAG1^{-/-} HSCs (FIG. 1B).

Next, cells in the thymus were examined. It was determined that 5% of the thymocytes in B6/MOT1 mice and 3% of the thymocytes in B6/MOT2 mice expressed the transgenic OT1 or OT2 T cell receptors, respectively. This result indicates that these cells were derived from the transduced HSCs (FIG. 1C, upper right). Study of the surface expression pattern of the developmental markers CD4 and CD8 showed that the CD8 SP compartment in B6/MOT1 and B6/MOT2 mice were selectively enriched compared to B6 controls (4.3% for B6/MOT1 mice compared to 2.0% in B6 control mice and 8.3% for B6/MOT2 mice compared to 4.5% in B6 control mice; FIG. 1C, lower right). This result indicates that expression of OT1 or OT2 T cell receptor transgenes in thymocytes can direct them to the appropriate T cell fate.

Finally, cells in the periphery were analyzed. In the spleen, about 25% of the CD8 T cells in B6/MOT1 mice were OT1 T cells, and no CD4 T cells expressed OT1 T cell receptors (FIG. 1D, upper right). On the other hand, in the spleen of B6/MOT2 mice, about 8% of the CD4 T cells were OT2 T cells. No CD8 T cells expressed OT2 T cell receptors (FIG. 1D, lower right). These results demonstrate that retrovirus-mediated transfer of T cell receptor cDNAs into wild-type HSCs is highly efficient in generating T cells with the desired characteristic (CD4 vs. CD8 T cells) and specificity.

### Example 5

### Characterization of the CD8 and CD4 T Cells Generated by Viral Transduction of Wild-Type HSCs

This Example demonstrates that the OT1 CD8 T cells and the OT2 CD4 T cell generated using retrovirus transduction of B6 HSCs are normal and fully functional as CD8 and CD4 T cells respectively.

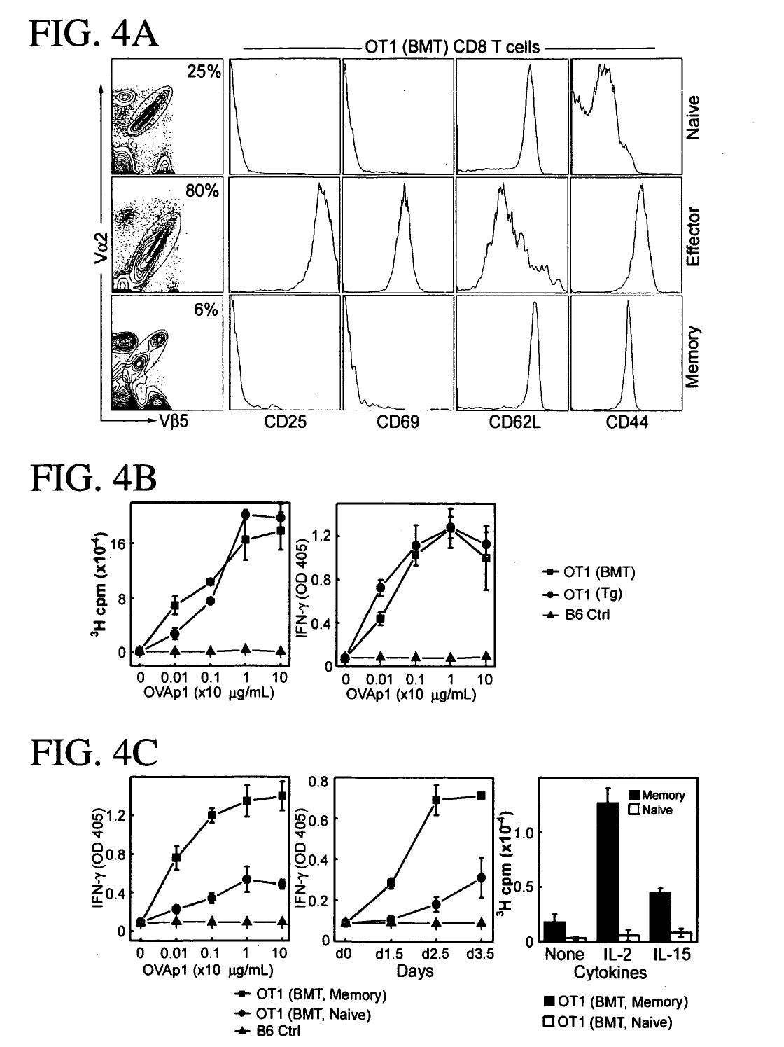

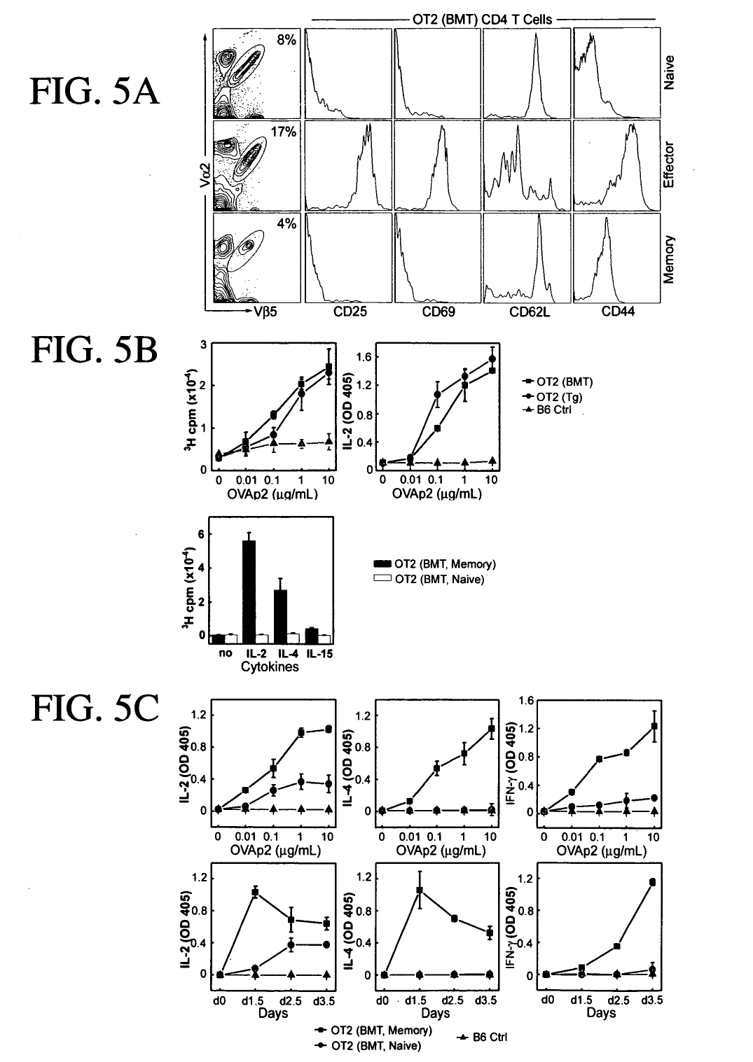

The functionality of CD8 and CD4 T cells generated by viral transduction of wild-type HSCs was tested. First, the OT1 CD8 T cells generated by MOT1-mediated bone marrow transfer were investigated. Spleen cells were harvested from B6/MOT1 mice and stimulated with OVAp1 in culture. Before stimulation, about 25% of the CD8 T cells were OT1 cells that showed a naïve CD8 T cell phenotype of CD25⁻CD69⁻CD62L^{high}CD44^{low} as measured by surface staining (FIG. 4A, upper). After stimulation with OVAp1 for 3 days, OT1 T cells expanded to 80% of the total CD8 T cells in the culture (FIG. 4A, middle). Study of the surface activation markers showed that these activated OT1 T cells expressed a typical effector CD8 T cell phenotype: CD25^{high}CD69^{high}CD62^{low}CD44^{high} (FIG. 4A, middle). When compared with OT1 T cells harvested from the conventional T cell receptor transgenic mice (designated as OT1(Tg)), the OT1 T cells generated by retroviral mediated BM transfer (designated as OT1(BMT)) showed comparable proliferation (FIG. 4B, left) and IFN-γ production (FIG. 4B, middle) in response to antigenic stimulation.

A unique feature of the adaptive immune system is the ability to generate long-term memory after the initial antigen encounter, thus providing more efficient protection for the next infection. In this regard, the ability of OT1 (BMT) T cells to generate memory was tested. Effector OT1 T cells were collected from culture after stimulation with OVAp1 for 3 days and the activated cells were adoptively transferred into RAG1^{-/-} recipients. Sixteen weeks later, the recipients were analyzed for the presence of long-lived memory OT1 T cells. As shown in FIG. 4A (lower), about 6% of the recovered CD8 T cells were OT1 T cells. Surface staining of the activation markers showed that these OT1 T cells expressed the memory T cell phenotype: CD25⁻CD69⁻CD62L^{high}CD44^{high} (FIG. 4A, lower right). Furthermore, when stimulated with OVAp1 in the culture, these OT1 T cells showed a larger and faster response as measured by IFN-γ production, compared with the response of the naïve OT1 T cells (FIG. 4C, left and middle). Finally, cytokine-induced proliferation was measured. This is a unique feature of memory T cells because the proliferation of naïve T cells is strictly controlled by antigen recognition. When stimulated with the cytokines IL-2 and IL-15, these OT1 T cells responded with extensive proliferation, while the naïve OT1 T cells did not (FIG. 4C, right).

In addition, the functionality of the OT2 T cells generated by MOT2 mediated BM transfer (designated as OT2(BMT)) was tested. Spleen cells were harvested from B6/MOT2 mice and stimulated with OVAp2 in the culture. Before stimulation, about 8% of the spleen CD4 T cells expressed OT2 T cell receptors (FIG. 4D, upper left). Surface staining showed that these OT2 CD4 T cells were of the native T cell phenotype: CD25⁻CD69⁻CD62^{high}CD44^{low} (FIG. 4D, upper right). After stimulation with OVAp2 for 3 days, the OT2 T cells expanded to 17% of the total CD4 T cells in culture, and expressed the typical effector CD4 T cell phenotype: CD25^{high}CD69^{high}CD62L^{low}CD44^{high} (FIG. 4D, middle).

When compared with OT2 T cells harvested from the conventional OT2 T cell receptor transgenic mice (designated as OT2(Tg)), the OT2(BMT) cells showed comparable proliferation and IL-2 production (FIG. 4E, left and middle). The ability of these OT2 T cells to generate long-term memory was also tested. Effector OT2 CD4 T cells were collected from culture after stimulation with OVAp2 for 3 days and adoptively transferred into RAG1 recipients. Fourteen weeks later, the recipients were analyzed. As shown in FIG. 4D (lower left), the presence of long-lived OT2 T cells (˜4% of the total CD4 T cells) was detected. These OT2 T cells displayed the memory phenotype of CD25⁻CD69⁻CD62L^{high}CD44^{high} (FIG. 4D, lower right). Compared with native OT2 T cells, these OT2 T cells showed a stronger response to antigen stimulation (FIG. 4F, upper) and a faster response (FIG. 4F, lower), as measured by IL-2, IL-4 and IFN-γ production. Moreover, these OT2 T cells proliferated intensively when stimulated with the cytokines IL2, IL4 and IL-15, while the naïve OT2 T cells did not (FIG. 4E, right).

Taken together, these results reveal that the OT1 CD8 T cells and the OT2 CD4 T cells generated using retrovirus transduction of B6 HSCs are normal and fully functional. In particular, these T cells can generate and maintain long-term memory, making them markedly attractive for immunotherapy.

### Example 6

### Imparting Both Anti-Tumor CD8 Cytotoxic and CD4 Helper T Cell Specificities into a Mouse T Cell Repertoire

This Example demonstrates that it is possible to cytotoxic T cells and helper T cells simultaneously in a host, thereby reducing the risk that epitope escape can occur. The methods disclosed herein using HSCs as the targets for gene transfer offers the opportunity to divide them into sub-pools and deliver different genes into each sub-pool. In the case of imparting anti-tumor specificities to the T cell repertoire, this allows for the production of both CD8 and CD4 T cells in vivo simultaneously.

In the model system, by dividing the HSCs into two sub-pools and transducing one with MOT1 and the other with MOT2 retroviruses, both OT1 CD8 and OT2 CD4 T cells were generated in vivo. Briefly, B6 mice were treated with 5-FU to enrich the HSCs. Five days later, BM cells were harvested and divided into two populations. One population of the cells was infected with MOT1 retroviruses and the other with MOT2 retroviruses. The transduced HSCs were then pooled together and transferred into irradiated B6 recipient mice (designated as B6/MOT1+MOT2). The recipients were allowed to reconstitute their immune system for 6 weeks and then were analyzed for the presence of OT1 and OT2 T cells.

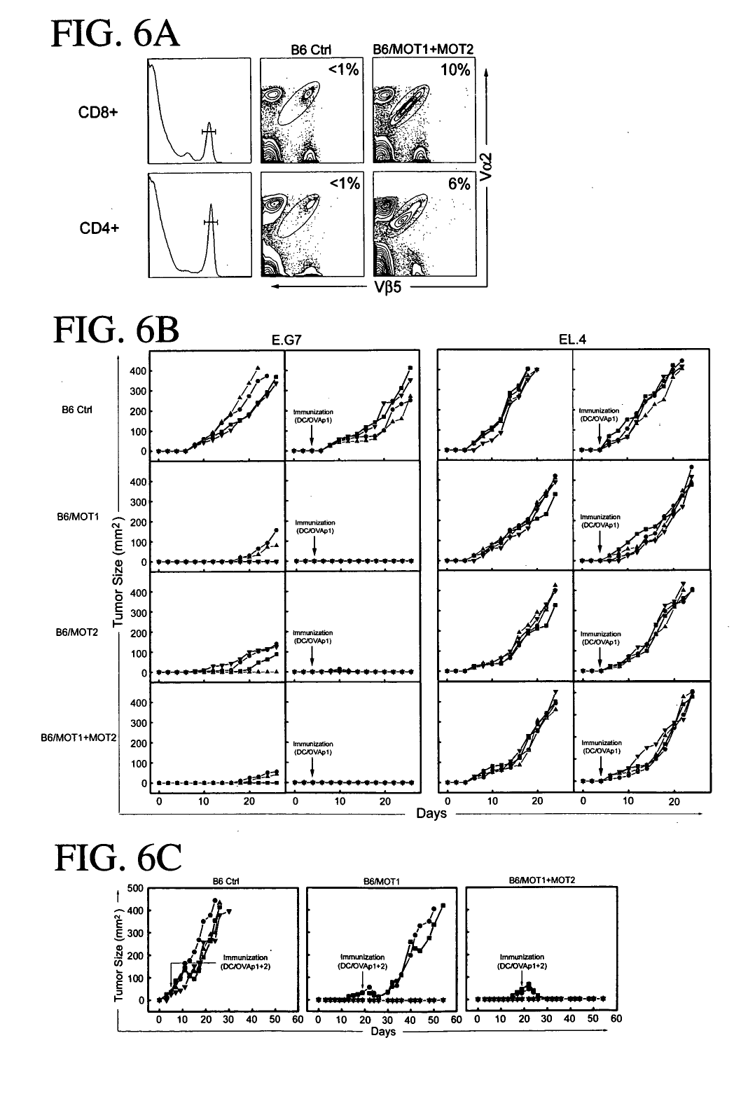

OT1 CD8 T cells and OT2 CD4 T cells were generated in the recipient mice, accounting for about 10% of the peripheral CD8 and 6% of the peripheral CD4 T cells, respectively (FIG. 6A). Further analysis showed that they exhibited completely normal functional characteristics of T cells. Therefore, the methods disclosed herein can be used to efficiently impart to the T cell repertoire both anti-tumor CD8 cytotoxic and CD4 helper T cell specificities.

### Example 7

### Imparting Multiple T Cell Specificities into a Mouse T Cell Repertoire

A further extension of the methods discussed in Example 6 is to impart to the T cell repertoire cytotoxic T cells and helper T cells that recognize multiple epitopes of a tumor antigen or multiple different antigens, thus providing a new opportunity to overcome the tendency of tumors towards “epitope escape.” This can be achieved by transfecting one-pool of target cells with a TCR that is specific for a first epitope and a second pool of target cells with a TCR that is specific for a second epitope. Upon transfer into a host, two sets of immune cells will develop, each specific for a different epitope associated with a disease antigen. In other embodiments, helper T cells and cytotoxic T cells are generated for each epitope or antigen. The number of antigens or epitopes to which immune cells can be generated is not limited.

### Example 8

### Tumor Immunotherapy: Suppression of Syngenic Tumor Growth by Imparting Anti-Tumor Specificities to the T Cell Repertoire

This Example demonstrates that tumors can be treated by generating antigen specific CD8 and CD4 T cells in vivo by retroviral transduction of HSCs. Additionally, this example demonstrates the advantages of using both arms of the immune system at once through this method, as well as the advantages of immunization with the particular TCR antigen.

To evaluate the anti-tumor function of each arm of the T cell immunity (CD8 CTLs and CD4 helper T cells) and the combination of both, mice imparted with anti-tumor CD8 specificity (B6 mice receiving B6 HSCs transduced with MOT1, designated as B6/MOT1), or anti-tumor CD4 specificity (B6 mice receiving B6 HSCs transduced with MOT2, designated as B6/MOT2) or both (B6 mice receiving both B6 HSCs transduced with MOT1 and HSCs transduced with MOT2, designated as B6/MOT1+MOT2) were utilized.

First, the suppression of syngenic tumor growth was evaluated. Briefly, B6 mice receiving B6 HSCs transduced with MOT1, MOT2 or a mixture of both were allowed to reconstitute the immune system for 8-10 weeks. E.G7 or the control tumor cells EL.4 were then injected subcutaneously. Each mouse received 5×10⁶ E.G7 or EL.4 tumor cells.

To evaluate the effects of immunization, 4 days after the tumor injection, 8 groups of mice out of 16 were immunized with one dose of dendritic cells (DCs) loaded with OVAp1 to boost anti-tumor CD8 response.

Tumor growth was monitored daily, and mice were euthanized when tumors reached the size of 400 mm². Four mice were used in each group and the experiments were performed at least three times.

A. CD8 Cells

Results from one representative experiment are shown in FIG. 6B. In B6 control mice that were not imparted with anti-tumor specificities, E.G7 tumors grew up at a rate similar to the control EL.4 tumor cells, resulting in visible solid tumors in one week. The control tumors reached a size of 400 mm² in about 3 weeks. In sharp contrast, E.G7 tumor growth was greatly suppressed in B6 mice imparted with anti-tumor CD8 T cell specificity (B6/MOT1 mice). In half of the B6/MOT1 mice, total tumor suppression was observed for as long as the experiment ran (up to 200 days).

For the other half of the B6/MOT1 mice, tumor growth was suppressed for about 18 days but then progressed. Consistently, OT1 T cells harvested from these tumor-bearing mice did not respond when stimulated with antigen in vitro, apparently having been attenuated by tumor tolerance mechanisms.

The ability of booster immunization to aid in tumor suppression by activating OT1 T cells was tested. With a single dose of immunization with dendritic cells (DCs) loaded with OVAp1, complete tumor suppression was observed for all the mice without recurrence for as long as the experiment ran (up to 200 days).

EL.4 tumor grew in B6/MOT1 mice at the same rate as in B6 control mice, regardless of immunization, indicating that the suppression of E.G7 tumor growth is tumor-antigen specific and is mediated by the anti-tumor OT1 T cells. B. CD4 Cells

Significant tumor suppression was also observed in mice imparted with anti-tumor CD4 specificity (B61MOT2 mice). As shown in FIG. 6B for a representative group, complete tumor suppression was observed in one out of four B6/MOT2 animals. In the other 3 mice tumor growth was suppressed for 10-20 days and then progressed. Study of the OT2 T cells recovered from the tumor-bearing mice showed that they could not respond to antigen stimulation in vitro, suggesting they had been subjected to the similar tumor tolerance mechanisms.

E.G7 tumor cells are MHC class II negative. Thus, OT2 T cells could probably not recognize and respond to them in vitro and the tumor suppression observed in B6/MOT2 mice was likely not mediated by the direct recognition of the E.G7 tumor cells by the OT2 T cells.

This phenomenon of CD4 T cell mediated suppression of MHC class II negative tumors has been reported in other cases, such as the FBL-3 murine leukemia tumor model (Pardoll and Topalian, 1998). The working hypothesis is that tumor antigens released at the tumor sites are ingested, processed and presented by macrophages. The tumor specific CD4 T cells recognize the tumor antigens, are activated and prime multiple arms of the anti-tumor immunity, including CTL activation, macrophage activation and eosinophil activation (Pardoll and Topalian, 1998).

In the B6/MOT2 mice, anti-tumor CTL activity plays an important role. When these mice were immunized with one dose of DC pulsed with OVAp1 (the epitope recognized by CD8 T cells) to activate the anti-tumor CTL response, total suppression of tumor growth was observed in half of the B6/MOT2 mice (FIG. 6B). In the other half of the mice, tumors grew up to a barely detectable size and soon regressed. In all the mice, no tumor recurrence was observed as long as the experiment went on (up to 200 days).

EL.4 tumors grew in B6/MOT2 mice at the same rate as in the B6 control mice with or without immunization, indicating that the tumor suppression observed in these mice is tumor antigen specific and mediated by the imparted OT2 CD4 T cell anti-tumor specificity (FIG. 6B, right).

C. CD4 and CD8 Cells Combined

When mice imparted with both anti-tumor CD8 and CD4 T cell specificities (B6/MOT1+MOT2) were analyzed, a combinatory effect was observed. As shown in FIG. 6B, complete tumor suppression was seen in half of the animals. For the other half, tumor growth was suppressed for about 18 days and then progressed, but at a rate slower than that observed in B6/MOT1 mice or B6/MOT2 mice (FIG. 6B, far left). As a result, it took longer for the tumors to reach a size of 400 mm² (about 50 days after tumor challenge) in the B6/MOT1+MOT2 mice than the tumor-bearing mice in the B6/MOT1 or B6/MOT2 groups (about 36-38 days after tumor challenge). Furthermore, obvious lesions were observed on most of the tumors, suggesting the presence of active anti-tumor immunity. Nevertheless, the final progress of the tumors in half of the animals indicated the existence of tumor tolerance, which was confirmed by a much reduced response to antigen stimulation in vitro of the OT1 and OT2 T cells recovered from these mice. These results indicate that imparting to the T cell repertoire both anti-tumor CD8 and CD4 T cell specificities has an advantage in anti-tumor immunotherapy over imparting only one of the two arms.

Immunization of the B6/MOT1+MOT2 mice with one dose of DCs loaded with OVAp1 completely suppressed E.G7 tumor growth (FIG. 6B). Tumor growth was suppressed for as long as the experiment was continued.

In the control, EL.4 tumors grew at the same rate in B6/MOT1+MOT2 mice as in B6 control mice, regardless of immunization (FIG. 6B, right). This indicates that the E.G7 tumor suppression is tumor-specific and mediated by the anti-tumor OT1 CD8 and OT2 CD4 T cell specificities imparted into the B6/MOT1+MOT2 mice.

### Example 9

### Eradication of Established Solid Tumors by Reversal of Functional Tumor Tolerance Via Construction of the Two Arms of Anti-Tumor T Cell Immunity

This Example demonstrates that solid tumors can be eradicated in a host by constructing both arms of the anti-tumor T cell immunity by the disclosed methods. The experiment included B6 control mice, B6 mice imparted with CTL anti-tumor specificity (B6/MOT1) and B6 mice imparted with both CD8 CTL and CD4 helper T cell anti-tumor specificities (B6/MOT1+MOT2).

Mice that received BM transfer were allowed to reconstitute the immune system for 6-10 weeks. The mice were then challenged with E.G7 tumor cells subcutaneously. Each mouse received 1×10⁶ E.G7 tumor cells. Mice in which tumors grew were immunized with one dose of DCs loaded with both OVAp1, the epitope recognized by OT1 T cell receptor, and OVAp2, the epitope recognized by OT2 T cell receptor. Immunizations were performed when the tumors reached the size of 30 mm².

Tumor growth was monitored daily and mice were euthanized when tumors reached the size of 400 mm². Four mice were used in each group and the experiments were performed three times.

Results from one representative experiment are shown in FIG. 6C. In B6 control mice, E.G7 tumors grew in 3 days and reached the size of about 30 mm² at day 5. The mice were then immunized with one dose of DCs loaded with OVAp1 and OVAp2. Tumor growth was not affected and continued to progress, with tumors reaching the size of 400 mm² in 20-24 days (FIG. 6C, left), confirming that suppression of tumor growth was tumor antigen specific and mediated by the engineered anti-tumor CD8 and/or CD4 T cells.

In B6/MOT1 and B6/MOT1+MOT2 mice, complete tumor suppression was seen in half of the mice (FIG. 6C) for as long as the experiment ran (up to 150 days) as was observed previously (FIG. 6B). For the other half of the mice, tumors were suppressed for 14-18 days and then progressed (FIG. 6C, middle and right), consistent with the observation reported in FIG. 6B.

By combining the two arms of anti-tumor T cell immunity, established, large vascularized solid tumors can be eradicated. On day 18, when tumors were about 30 mm² in size, each tumor bearing mouse was immunized with one dose of DCs loaded with OVAp1 and OVAp2. In B6/MOT1 mice bearing tumors, the tumor was suppressed and remained below the size of 50 mm² until day 30, but then grew and reached the size of 400 mm² in about 50 days (FIG. 6C, middle). In sharp contrast, for B6/MOT1+MOT2 mice bearing tumors, the tumors shrank after the immunization and totally disappeared by day 32 (FIG. 6C, right). These mice remained tumor-free, with no tumor recurrence observed in the majority of them for as long as the experiment ran (greater than 200 days). In a few cases, tumor recurrence was observed after 90 days. However, multiple immunizations completely prevented tumor recurrence in all animals.

These results confirmed that imparting to the T cell repertoire both arms of the anti-tumor T cell immunity, combined with immunization to activate both arms, can eradicate solid vascularized tumors.

### Example 10

### Treatment of Tumors in a Patient Via Construction of the Two Arms of Anti-Tumor T Cell Immunity

An antigen is identified that is associated with a tumor from which a patient is suffering. A first T cell receptor is cloned from a cytotoxic T cell that can bind to the antigen of interest. When a TCR that associates with a MHC I (or a CD8) and binds to the antigen is known, it can be used, rather than requiring the cloning of the TCR. A second T cell receptor, also specific for the antigen is cloned from a helper T cell. Again, when there is a known TCR that associates with a MHC II (or a CD4) and binds to the antigen, it can be used rather than requiring the cloning of the TCR. Hematopoietic stem cells, preferably bone marrow stem cells, are obtained from the patient. For example, the stem cells may be obtained during chemotherapy. Stem cells are fractionated into pools and one pool is transfected with the first T cell receptor that was obtained from a cytotoxic T lymphocyte and a second pool of stem cells is transfected with the T cell receptor that was obtained from the helper T cell. The two pools of transfected T cells are then transferred back into the patient by injection. The transfected stem cells mature into a population of cytotoxic T cells and helper T cells that are specific for the antigen associated with the tumor in the patient.

Optionally, the patient can be administered at least one immunization comprising the antigen to the TCRs. The actual immunization can comprise antigens or epitopes for both TCRs. The immunization can be repeated indefinitely to provide a prolonged beneficial effect from the treatment. In a preferred embodiment, dendritic cells are obtained from the patient and expanded in culture. The disease associated antigen is obtained, for example by purification from tumor tissue or by synthesis. The dendritic cells are loaded with the antigen and injected with the patient. The injections are preferably repeated at regular intervals until the tumor has been eradicated.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments but is instead to be defined by reference to the appended claims.

1. A method of treating a disease in a patient comprising:
providing hematopoietic stem cells transfected with a vector encoding the α and β chains of a T cell receptor that is specific for an antigen associated with the disease;
transferring the transfected stem cells into the patient; and
immunizing the patient with the disease-associated antigen.

2. The method of claim 1, wherein the T cell receptor is from a cytotoxic T lymphocyte.

3. The method of claim 1, wherein the T cell receptor is from a helper T cell.

4. The method of claim 1, wherein immunizing comprises injecting the patient with antigen presenting cells comprising the tumor-associated antigen.

5. The method of claim 4, wherein the antigen presenting cells are dendritic cells.

6. The method of claim 5, wherein the dendritic cells were obtained from the patient.

7. The method of claim 1, wherein immunizing is carried out at least one day following transfer of the transfected stem cells into the patient.

8. The method of claim 1, wherein the disease is selected from the group consisting of cancer and a viral infection.

9. The method of claim 8, wherein the cancer comprises a tumor.

10. The method of claim 1, wherein the vector is a retroviral vector.

11. The method of claim 10, wherein the retroviral vector is a lentiviral vector.

12. The method of claim 1, wherein the vector comprises a first cDNA encoding the α chain of the T cell receptor and a second cDNA encoding the β chain of the T cell receptor.

13. The method of claim 12, wherein the first and second cDNAs are separated by an IRES element.

14. The method of claim 1, wherein the hematopoietic stem cells are primary bone marrow cells.

15. A method of treating a disease in a patient comprising:
providing a first population of target cells transfected with a polynucleotide encoding a first T cell receptor from a cytotoxic T cell;
providing a second population of target cells transfected with a polynucleotide encoding a second T cell receptor from a helper T cell;
transferring the first and second populations of transfected target cells into the patient,
wherein the first and second T cell receptors are specific for an antigen associated with the disease.

16. The method of claim 15, additionally comprising:
providing a third population of target cells transfected with a polynucleotide encoding a third T cell receptor; and
transferring the third population of transfected target cells into the patient,
wherein the third T cell receptor is specific for a different antigen associated with the disease.

17. The method of claim 16, wherein the third T cell receptor is from a cytotoxic T cell or a helper T cell.

18. The method of claim 15, additionally comprising immunizing the patient with the disease-associated antigen.

19. The method of claim 18, wherein immunizing is carried out at least one day following transfer of the first and second populations of transfected target cells into the patient.

20. The method of claim 18, wherein immunizing comprises injecting the patient with the disease-associated antigen.

21. The method of claim 18, wherein immunizing comprises injecting the patient with dendritic cells comprising the tumor associated antigen.

22. The method of claim 21 wherein the dendritic cells were obtained from the patient.

23. The method of claim 18, wherein the immunization is repeated two or more times.

24. The method of claim 15, wherein the disease is selected from the group consisting of cancer and a viral infection.

25. The method of claim 24, wherein the cancer comprises a tumor.

26. The method of claim 15, wherein the target cells comprise hematopoietic stem cells.

27. The method of claim 26, wherein the hematopoietic stem cells are primary bone marrow cells.

28. The method of claim 26, wherein the hematopoietic stem cells are obtained from the patient.

29. The method of claim 26, wherein the hematopoietic stem cells are obtained from an immunologically compatible donor.

30. A method of generating in a mammal cytotoxic T cells and helper T cells responsive to an antigen of interest, the method comprising:
transfecting a first population of hematopoietic stem cells with a first vector encoding the α and β chains of a first T cell receptor from a cytotoxic T cell; and
transfecting a second population of hematopoietic stem cells with a second vector encoding the α and β chains of a second T cell receptor from a helper T cell, wherein the first and second T cell receptors are specific for the antigen of interest

31. The method of claim 30, wherein the hematopoietic stem cells are obtained from the mammal.

32. The method of claim 30, additionally comprising transferring the first and second populations of transfected stem cells to the mammal.
